(19) United States
(12) Patent Application Publication
(10) Pub. No.: US 2022/0192619 A1
(43) Pub. Date: Jun. 23, 2022

(54) IMAGING SYSTEMS AND METHODS

(71) Applicant: SHANGHAI UNITED IMAGING HEALTHCARE CO., LTD., Shanghai (CN)

(72) Inventor: Haohua Sun, Shanghai (CN)

(73) Assignee: SHANGHAI UNITED IMAGING HEALTHCARE CO., LTD., Shanghai (CN)

(21) Appl. No.: 17/645,460

(22) Filed: Dec. 22, 2021

(30) Foreign Application Priority Data
Dec. 22, 2020 (CN) 202011527062.X

(51) Int. Cl. A61B 6/00; A61B 5/055; A61B 8/08; A61B 8/00; G16H 30/40; G16H 50/50

(52) U.S. Cl. CPC A61B 6/545; A61B 5/055; A61B 6/5247; A61B 6/032; A61B 8/54; G16H 30/40; G16H 50/50; A61B 8/5261

(57) ABSTRACT

The present disclosure is related to systems and methods for imaging. The method includes obtaining feature information of a target subject. The method includes obtaining target prior information of the target subject based on the feature information of the target subject and a prior information database. The prior information database includes prior information of a plurality of candidate subjects. The method includes determining, based on the target prior information of the target subject, a value of a procedure parameter that relates to a procedure of the target subject using a medical device

### CROSS-REFERENCE TO RELATED APPLICATIONS

This application claims priority of Chinese Patent Application No. 202011527062.X, filed on Dec. 22, 2020, the contents of which are hereby incorporated by reference.

### TECHNICAL FIELD

The present disclosure generally relates to a medical system, and more particularly, relates to systems and methods for determining a value of a procedure parameter related to a procedure of a target subject.

### BACKGROUND

Medical systems, such as a CT device, an MRI device, a PET device, are widely used for generating images of the interior of a patient for medical diagnosis and/or treatment purposes. Generally, one or more procedure parameters (e.g., an imaging scan parameter, an image reconstruction parameter) of a patient may be determined and/or adjusted based on a scan protocol of the patient. However, in some cases, to ensure a successful scan in which imaging data of sufficient quality is generated, the dose of the imaging medium (e.g., radiation) may be increased and/or scanning time may be extended, thereby causing unnecessary exposure to excessive imaging medium and/or discomfort from the extended scanning time. Therefore, it is desired to provide systems and methods for determining a value of the procedure parameter efficiently and accurately, thereby obliviating or reducing an unnecessary radiation dose boost during a scan of a patient.

### SUMMARY

According to an aspect of the present disclosure, a method may be implemented on a computing device having one or more processors and one or more storage devices. The method may include obtaining feature information of a target subject. The method may include obtaining target prior information of the target subject based on the feature information of the target subject and a prior information database. The prior information database may include prior information of a plurality of candidate subjects. The method may include determining, based on the target prior information of the target subject, a value of a procedure parameter that relates to a procedure of the target subject using a medical device.

In some embodiments, the procedure parameter may include an imaging scan parameter and an image reconstruction parameter.

In some embodiments, the method may include obtaining scan data of the target subject by causing the medical device to scan the target subject based on a value of the imaging scan parameter. The method may include generating an image of the target subject based on the scan data and a value of the image reconstruction parameter.

In some embodiments, a first modality corresponding to scan data obtained by of the medical device may be the same as a second modality corresponding to the target prior information. The target prior information may include a candidate value of the imaging scan parameter of the target subject. The method may include determining a plurality of simulation values of the imaging scan parameter based on the candidate value of the imaging scan parameter. The method may include generating a plurality of simulated images based on the plurality of simulation values of the imaging scan parameter and a plurality of simulation values of the image reconstruction parameter. The method may include determining the value of the procedure parameter based on the plurality of simulated images.

In some embodiments, the method may include obtaining an initial value of the imaging scan parameter. The method may include obtaining an initial image based on the initial value of the imaging scan parameter and the candidate value of the imaging scan parameter using a dose simulation model. The method may include determining at least one of the plurality of simulation values of the imaging scan parameter based on the initial image and the initial value of the imaging scan parameter.

In some embodiments, the dose simulation model may be obtained according to a process. The process may include obtaining a plurality of training samples each of which includes a sample value of the imaging scan parameter, a sample image corresponding to the sample value of the imaging scan parameter, a reference value of the imaging scan parameter, and a reference image corresponding to the reference value of the imaging scan parameter. The process may include determining the dose simulation model by training a preliminary model based on the plurality of training samples.

In some embodiments, the target prior information may include candidate scan data corresponding to the candidate value of the imaging scan parameter of the target subject. The method may include, for each simulation value of the plurality of simulation values of the imaging scan parameter, determining simulation scan data based on the candidate scan data, the candidate value of the imaging scan parameter, and the simulation value of the imaging scan parameter. The method may include, for each simulation value of the plurality of simulation values of the image reconstruction parameter, generating a simulated image based on the simulation scan data and the simulation value of the image reconstruction parameter.

In some embodiments, a first modality of the medical device may be different from a second modality corresponding to the target prior information. The method may include generating a first image of the first modality based on a second image of the second modality in the target prior information.

In some embodiments, the first modality or the second modality may include at least one of an ultrasound imaging, an X-ray imaging, a computed tomography (CT), a magnetic resonance imaging (MRI), a single photon emission computed tomography (SPECT), or a positron emission tomography (PET).

In some embodiments, the method may include determining a dimension of a phantom corresponding to the target subject based on the feature information of the target subject and the prior information database.

In some embodiments, the dimension of the phantom corresponding to the target subject may include a target water equivalent diameter of the phantom corresponding to the target subject.

In some embodiments, the prior information database may include candidate scan data of the target subject. The method may include determining the target water equivalent diameter based on the candidate scan data of the target subject.

In some embodiments, the prior information database may include a topogram image of the target subject. The method may include determining the target water equivalent diameter based on the topogram image of the target subject.

In some embodiments, the method may include obtaining a plurality of candidate images each of which is acquired by a simulated scanning, based on one of a plurality of present values of the imaging scan parameter, of one of a plurality of phantoms of a preset water equivalent diameter. The method may include determining a plurality of target values of the imaging scan parameter based on the plurality of candidate images, a plurality of preset water equivalent diameters, and the plurality of preset values of the imaging scan parameter. The method may include selecting a value of the imaging scan parameter from the plurality of target values of the imaging scan parameter.

In some embodiments, the target prior information may include a recommended value of the procedure parameter. The method may include determining whether the recommended value of the procedure parameter satisfies a scan condition of the target subject. The method may include, in response to determining that the recommended value of the procedure parameter satisfies the scan condition, determining the value of the procedure parameter based on the recommended value of the procedure parameter.

In some embodiments, the prior information database may be established based on at least one of feature information of a candidate subject, a historical scan protocol of the candidate subject, a historical value of an imaging scan parameter of the candidate subject, a historical value of an image reconstruction parameter of the candidate subject, historical scan data of the candidate subject, a historical image of the candidate subject, a simulation value of the imaging scan parameter of the candidate subject, a simulation value of the image reconstruction parameter of the candidate subject, simulation scan data of the candidate subject, or a simulated image of the candidate subject.

According to another aspect of the present disclosure, a system may include at least one storage device storing a set of instructions, and at least one processor in communication with the at least one storage device. When executing the stored set of instructions, the at least one processor may cause the system to perform a method. The method may include obtaining feature information of a target subject. The method may include obtaining target prior information of the target subject based on the feature information of the target subject and a prior information database. The prior information database may include prior information of a plurality of candidate subjects. The method may include determining, based on the target prior information of the target subject, a value of a procedure parameter that relates to a procedure of the target subject using a medical device. The procedure parameter may include an imaging scan parameter and an image reconstruction parameter. The method may include obtaining scan data of the target subject by causing the medical device to scan the target subject based on a value of the imaging scan parameter. The method may include generating an image of the target subject based on the scan data and a value of the image reconstruction parameter.

According to another aspect of the present disclosure, a non-transitory computer readable medium may include at least one set of instructions. When executed by at least one processor of a computing device, the at least one set of instructions may cause the at least one processor to effectuate a method. The method may include obtaining feature information of a target subject. The method may include obtaining target prior information of the target subject based on the feature information of the target subject and a prior information database. The prior information database may include prior information of a plurality of candidate subjects. The method may include determining, based on the target prior information of the target subject, a value of a procedure parameter that relates to a procedure of the target subject using a medical device.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary medical system according to some embodiments of the present disclosure;

FIG. 2 is a schematic diagram illustrating exemplary hardware and/or software components of an exemplary computing device according to some embodiments of the present disclosure;

FIG. 3 is a schematic diagram illustrating exemplary hardware and/or software components of an exemplary mobile device according to some embodiments of the present disclosure;

FIG. 4 is a block diagram illustrating an exemplary processing device according to some embodiments of the present disclosure;

FIG. 5 is a flowchart illustrating an exemplary process for imaging according to some embodiments of the present disclosure;

FIG. 6 is a flowchart illustrating an exemplary process for determining a value of a procedure parameter according to some embodiments of the present disclosure; and

FIG. 7 is a flowchart illustrating an exemplary process for determining a value of a procedure parameter according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is to describe particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the terms “system,” “unit,” “module,” and/or “block” used herein are one method to distinguish different components, elements, parts, sections, or assemblies of different levels in ascending order. However, the terms may be displaced by another expression if they achieve the same purpose.

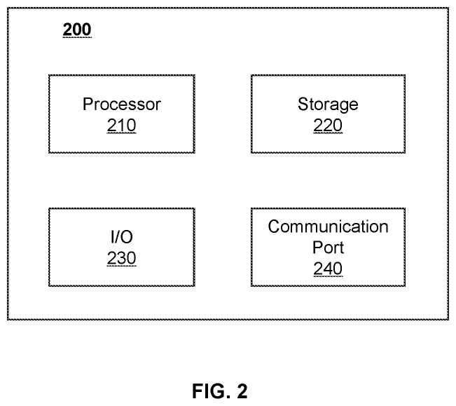

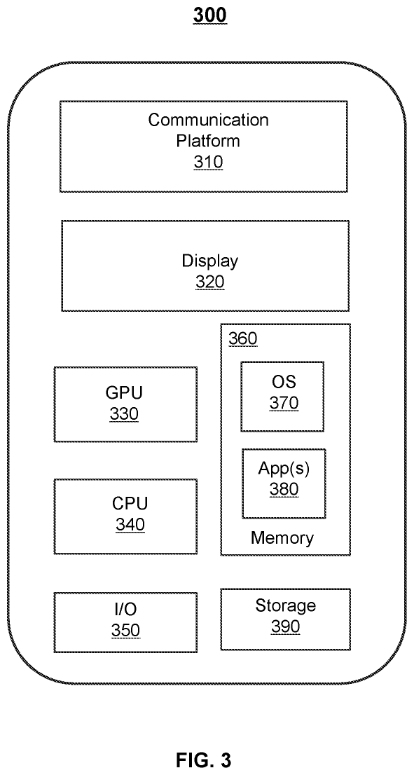

Generally, the words “module,” “unit,” or “block,” as used herein, refer to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices (e.g., the processor 210 illustrated in FIG. 2 and/or the central processing unit (CPU) 340 illustrated FIG. 3) may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may apply to a system, an engine, or a portion thereof.

It will be understood that when a unit, engine, module or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The flowcharts used in the present disclosure illustrate operations that systems implement according to some embodiments of the present disclosure. It is to be expressly understood, the operations of the flowcharts may be implemented not in order. Conversely, the operations may be implemented in inverted order, or simultaneously. Moreover, one or more other operations may be added to the flowcharts. One or more operations may be removed from the flowcharts.

Provided herein are medical systems and methods for non-invasive biomedical imaging/treatment, such as for disease diagnostic, disease therapy, or research purposes. In some embodiments, the medical system may include a single modality system and/or a multi-modality system. The term “modality” used herein broadly refers to an imaging or treatment method or technology that gathers, generates, processes, and/or analyzes imaging information of a subject or treatments the subject. The single modality system may include, for example, an ultrasound imaging system, an X-ray imaging system (e.g., a digital radiography (DR) system, a computed radiography (CR) system), a computed tomography (CT) system, a magnetic resonance imaging (MRI) system, an ultrasonography system, a single photon emission computed tomography (SPECT), a positron emission tomography (PET) system, an optical coherence tomography (OCT) imaging system, an ultrasound (US) imaging system, an intravascular ultrasound (IVUS) imaging system, a near-infrared spectroscopy (NIRS) imaging system, a digital subtraction angiography (DSA) system, or the like, or any combination thereof. The multi-modality system may include, for example, an X-ray imaging-magnetic resonance imaging (X-ray-MRI) system, a positron emission tomography-X-ray imaging (PET-X-ray) system, a single photon emission computed tomography-magnetic resonance imaging (SPECT-MRI) system, a positron emission tomography-computed tomography (PET-CT) system, a C-arm system, a positron emission tomography-magnetic resonance imaging (PET-MR) system, a digital subtraction angiography-magnetic resonance imaging (DSA-MRI) system, etc. In some embodiments, the medical system may include a treatment system. The treatment system may include a treatment plan system (TPS), an image-guided radiotherapy (IGRT) system, etc. The image-guided radiotherapy (IGRT) may include a treatment device and an imaging device. The treatment device may include a linear accelerator, a cyclotron, a synchrotron, etc., configured to perform radiotherapy on a subject. The treatment device may include an accelerator of species of particles including, for example, photons, electrons, protons, or heavy ions. The imaging device may include an MRI scanner, a CT scanner, etc. It should be noted that the medical system described below is merely provided for illustration purposes, and not intended to limit the scope of the present disclosure.

In the present disclosure, the term “image” may refer to a two-dimensional (2D) image, a three-dimensional (3D) image, or a four-dimensional (4D) image. In some embodiments, the term “image” may refer to an image of a region (e.g., a region of interest (ROI)) of a subject. As described above, the image may be a CT image, a PET image, an MR image, a fluoroscopy image, an ultrasound image, an Electronic Portal Imaging Device (EPID) image, etc.

As used herein, a representation of an object (e.g., a patient, a subject, or a portion thereof) in an image may be referred to as an “object” for brevity. For instance, a representation of an organ or tissue (e.g., a heart, a liver, a lung) in an image may be referred to as an organ or tissue for brevity. Further, an image including a representation of an object may be referred to as an image of an object or an image including an object for brevity. Still further, an operation performed on a representation of an object in an image may be referred to as an operation performed on an object for brevity. For instance, a segmentation of a portion of an image including a representation of an organ or tissue from the image may be referred to as a segmentation of an organ or tissue for brevity.

An aspect of the present disclosure relates to systems and methods for imaging. A processing device may obtain feature information of a target subject (e.g., a patient). The processing device may obtain target prior information of the target subject based on the feature information of the target subject and a prior information database. The prior information database may include prior information of a plurality of candidate subjects. The processing device may determine, based on the target prior information of the target subject, a value of a procedure parameter (e.g., a value of an imaging scan parameter, a value of an image reconstruction parameter) that relates to a procedure (e.g., a CT scan, an MRI scan, a PET scan) of the target subject using a medical device. In some embodiments, the processing device may obtain scan data of the target subject by causing the medical device to scan the target subject based on a value of the imaging scan parameter. The processing device may generate an image of the target subject based on the scan data and a value of the image reconstruction parameter. In some embodiments, in the present disclosure, “a value of a procedure parameter” may refer to value(s) of one or more procedure parameters. For example, in the present disclosure, “a value of an imaging scan parameter” may refer to value(s) of one or more imaging scan parameters, and “a value of an image reconstruction parameter” may refer to value(s) of one or more image reconstruction parameters. In some embodiments, the processing device may determine values of a plurality of procedure parameters based on the target prior information of the target subject.

Accordingly, the value of the procedure parameter (e.g., the value of the imaging scan parameter, the value of the image reconstruction parameter) of the target subject may be determined based on historical data (e.g., historical scan data, a historical image, a historical value of the procedure parameter) associated with one or more historical scans of the target subject and/or simulation data (e.g., simulation scan data, a simulated image, a simulation value of the procedure parameter) of the target subject stored in the prior information database, which may improve the efficiency, accuracy, and/or reliability of the parameter determination process. Moreover, the process may be automated, thereby reducing user involvement and/or inter-user variability in the parameter determination. In addition, the target subject may be scanned based on the value of the imaging scan parameter, and the image of the target subject may be generated based on the value of the image reconstruction parameter, which may reduce a radiation dose while maintaining a desired image quality acquired based on a scan. Furthermore, data (e.g., scan data, the value of the procedure parameter, the image generated based on the scan data) generated during new scans may be used to continuously update and/or optimize the prior information database, thereby further improving the efficiency and/or accuracy of the value of the procedure parameter determined using the prior information database. The improved procedure parameter determined according to some embodiments of the present disclosure may in turn improve the efficiency, accuracy, and/or efficacy of the procedure performed based on the determined procedure parameter.

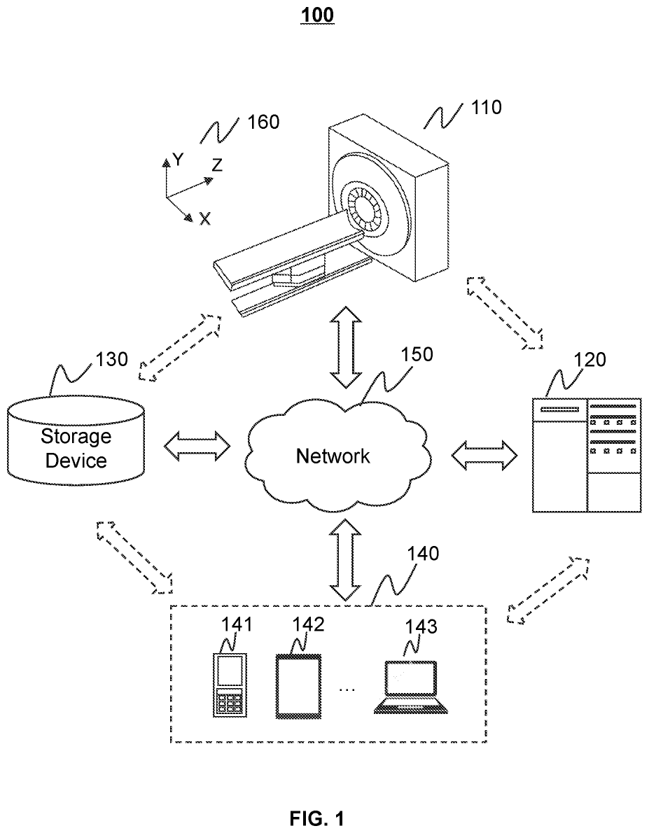

FIG. 1 is a schematic diagram illustrating an exemplary medical system according to some embodiments of the present disclosure. As illustrated in FIG. 1, the medical system 100 may include a medical device 110, a processing device 120, a storage device 130, a terminal device 140, and a network 150. In some embodiments, two or more components of the medical system 100 may be connected to and/or communicate with each other via a wireless connection, a wired connection, or a combination thereof. The medical system 100 may include various types of connection between its components. For example, the medical device 110 may be connected to the processing device 120 through the network 150, or connected to the processing device 120 directly as illustrated by the bidirectional dotted arrow connecting the medical device 110 and the processing device 120 in FIG. 1. As another example, the terminal device 140 may be connected to the processing device 120 through the network 150, or connected to the processing device 120 directly as illustrated by the bidirectional dotted arrow connecting the terminal device 140 and the processing device 120 in FIG. 1. As still another example, the storage device 130 may be connected to the medical device 110 through the network 150, or connected to the medical device 110 directly as illustrated by the bidirectional dotted arrow connecting the medical device 110 and the storage device 130 in FIG. 1. As still another example, the storage device 130 may be connected to the terminal device 140 through the network 150, or connected to the terminal device 140 directly as illustrated by the bidirectional dotted arrow connecting the terminal device 140 and the storage device 130 in FIG. 1.

The medical device 110 may be configured to acquire image data relating to a subject (e.g., a target subject). The image data relating to a subject may include an image (e.g., an image slice), projection data, or a combination thereof. In some embodiments, the image data may be two-dimensional (2D) image data, three-dimensional (3D) image data, four-dimensional (4D) image data, or the like, or any combination thereof. The subject may be biological or non-biological. For example, the subject may include a patient, a man-made object, etc. As another example, the subject may include a specific portion, an organ, and/or tissue of the patient. Specifically, the subject may include the head, the neck, the thorax, the heart, the stomach, a blood vessel, soft tissue, a tumor, or the like, or any combination thereof. In the present disclosure, “object” and “subject” are used interchangeably.

In some embodiments, the medical device 110 may include a single modality imaging device. For example, the medical device 110 may include a positron emission tomography (PET) device, a single-photon emission computed tomography (SPECT) device, a magnetic resonance imaging (MRI) device (also referred to as an MR device, an MR scanner), a computed tomography (CT) device (e.g., a spiral CT, an electron beam CT, an energy spectrum CT), an ultrasound (US) device, an X-ray imaging device, a digital subtraction angiography (DSA) device, a magnetic resonance angiography (MRA) device, a computed tomography angiography (CTA) device, or the like, or any combination thereof. In some embodiments, the medical device 110 may include a multi-modality imaging device. Exemplary multi-modality imaging devices may include a PET-CT device, a PET-MRI device, a SPET-CT device, or the like, or any combination thereof. The multi-modality imaging device may perform multi-modality imaging simultaneously. For example, the PET-CT device may generate structural X-ray CT data and functional PET data simultaneously in a single scan. The PET-MRI device may generate MRI data and PET data simultaneously in a single scan. In some embodiments, the medical device 110 may generate and emit imaging medium (e.g., radiation) toward the subject to perform a scan on the subject.

In some embodiments, the medical device 110 may transmit the image data via the network 150 to the processing device 120, the storage device 130, and/or the terminal device 140. For example, the image data may be sent to the processing device 120 for further processing or may be stored in the storage device 130.

The processing device 120 may process data and/or information. The data and/or information may be obtained from the medical device 110 or retrieved from the storage device 130, the terminal device 140, and/or an external device (external to the medical system 100) via the network 150. For example, the processing device 120 may obtain feature information of a target subject. As another example, the processing device 120 may obtain target prior information of a target subject based on feature information of the target subject and a prior information database. As still another example, the processing device 120 may determine, based on target prior information of a target subject, a value of a procedure parameter that relates to a procedure of the target subject using a medical device (e.g., the medical device 110). As still another example, the processing device 120 may obtain scan data of a target subject by causing a medical device (e.g., the medical device 110) to scan the target subject based on a value of an imaging scan parameter. As still another example, the processing device 120 may generate an image of a target subject based on scan data and a value of an image reconstruction parameter. In some embodiments, the processing device 120 may be a single server or a server group. The server group may be centralized or distributed. In some embodiments, the processing device 120 may be local or remote. For example, the processing device 120 may access information and/or data from the medical device 110, the storage device 130, and/or the terminal device 140 via the network 150. As another example, the processing device 120 may be directly connected to the medical device 110, the terminal device 140, and/or the storage device 130 to access information and/or data. In some embodiments, the processing device 120 may be implemented on a cloud platform. For example, the cloud platform may include a private cloud, a public cloud, a hybrid cloud, a community cloud, a distributed cloud, an inter-cloud, a multi-cloud, or the like, or any combination thereof. In some embodiments, the processing device 120 may be part of the terminal device 140. In some embodiments, the processing device 120 may be part of the medical device 110.

In some embodiments, the generation, and/or updating of a prior information database may be performed on a processing device, while the application of the prior information database may be performed on a different processing device. In some embodiments, the generation and/or updating of the prior information database may be performed on a processing device of a system different from the medical system 100 or a server different from a server including the processing device 120 on which the application of the prior information database is performed. For instance, the generation and/or updating of the prior information database may be performed on a first system of a vendor who provides and/or maintains such a prior information database, while parameter determination based on the provided prior information database may be performed on a second system of a client of the vendor. In some embodiments, the generation and/or updating of the prior information database may be performed on a first processing device of the medical system 100, while the application of the prior information database may be performed on a second processing device of the medical system 100. In some embodiments, the generation and/or updating of the prior information database may be performed online in response to a request for parameter determination or a request for a scan of a patient. In some embodiments, the generation and/or updating of the prior information database may be performed offline.

In some embodiments, the prior information database may be generated, and/or updated (or maintained) by, e.g., the manufacturer of the medical device 110 or a vendor. For instance, the manufacturer or the vendor may load the prior information database into the medical system 100 or a portion thereof (e.g., the processing device 120) before or during the installation of the medical device 110 and/or the processing device 120, and maintain or update the prior information database from time to time (periodically or not). The maintenance or update may be achieved by installing a program stored on a storage device (e.g., a compact disc, a USB drive, etc.) or retrieved from an external source (e.g., a server maintained by the manufacturer or vendor) via the network 150.

The storage device 130 may store data, instructions, and/or any other information. In some embodiments, the storage device 130 may store data obtained from the medical device 110, the processing device 120, and/or the terminal device 140. The data may include image data acquired by the processing device 120, algorithms and/or models for processing the image data, etc. For example, the storage device 130 may store a prior information database including prior information of a plurality of candidate subjects. As another example, the storage device 130 may store feature information of a target subject. As still another example, the storage device 130 may store target prior information of a target subject obtained from the prior information database. As still another example, the storage device 130 may store a value of a procedure parameter determined by the processing device 120. In some embodiments, the storage device 130 may store data and/or instructions that the processing device 120, and/or the terminal device 140 may execute or use to perform exemplary methods described in the present disclosure. In some embodiments, the storage device 130 may include a mass storage, a removable storage, a volatile read-and-write memory, a read-only memory (ROM), or the like, or any combination thereof. Exemplary mass storages may include a magnetic disk, an optical disk, a solid-state drive, etc. Exemplary removable storages may include a flash drive, a floppy disk, an optical disk, a memory card, a zip disk, a magnetic tape, etc. Exemplary volatile read-and-write memories may include a random-access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (EPROM), an electrically erasable programmable ROM (EEPROM), a compact disk ROM (CD-ROM), and a digital versatile disk ROM, etc. In some embodiments, the storage device 130 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the storage device 130 may be connected to the network 150 to communicate with one or more other components in the medical system 100 (e.g., the processing device 120, the terminal device 140). One or more components in the medical system 100 may access the data or instructions stored in the storage device 130 via the network 150. In some embodiments, the storage device 130 may be integrated into the medical device 110 or the terminal device 140.

The terminal device 140 may be connected to and/or communicate with the medical device 110, the processing device 120, and/or the storage device 130. In some embodiments, the terminal device 140 may include a mobile device 141, a tablet computer 142, a laptop computer 143, or the like, or any combination thereof. For example, the mobile device 141 may include a mobile phone, a personal digital assistant (PDA), a gaming device, a navigation device, a point of sale (POS) device, a laptop, a tablet computer, a desktop, or the like, or any combination thereof. In some embodiments, the terminal device 140 may include an input device, an output device, etc. The input device may include alphanumeric and other keys that may be input via a keyboard, a touchscreen (for example, with haptics or tactile feedback), a speech input, an eye tracking input, a brain monitoring system, or any other comparable input mechanism. Other types of the input device may include a cursor control device, such as a mouse, a trackball, or cursor direction keys, etc. The output device may include a display, a printer, or the like, or any combination thereof.

The network 150 may include any suitable network that can facilitate the exchange of information and/or data for the medical system 100. In some embodiments, one or more components of the medical system 100 (e.g., the medical device 110, the processing device 120, the storage device 130, the terminal device 140, etc.) may communicate information and/or data with one or more other components of the medical system 100 via the network 150. For example, the processing device 120 and/or the terminal device 140 may obtain feature information of a target subject from the medical device 110 via the network 150. As another example, the processing device 120 and/or the terminal device 140 may obtain a prior information database stored in the storage device 130 via the network 150. The network 150 may be and/or include a public network (e.g., the Internet), a private network (e.g., a local area network (LAN), a wide area network (WAN), etc.), a wired network (e.g., an Ethernet network), a wireless network (e.g., a Wi-Fi network), a cellular network (e.g., a long term evolution (LTE) network), a frame relay network, a virtual private network (VPN), a satellite network, a telephone network, routers, hubs, witches, server computers, and/or any combination thereof. For example, the network 150 may include a cable network, a wireline network, a fiber-optic network, a telecommunications network, an intranet, a wireless local area network (WLAN), a metropolitan area network (MAN), a public telephone switched network (PSTN), a Bluetooth™ network, a ZigBee™ network, a near field communication (NFC) network, or the like, or any combination thereof. In some embodiments, the network 150 may include one or more network access points. For example, the network 150 may include wired and/or wireless network access points such as base stations and/or internet exchange points through which one or more components of the medical system 100 may be connected to the network 150 to exchange data and/or information.

In some embodiments, a coordinate system 160 may be provided for the medical system 100 to define a position of a component (e.g., an absolute position, a position relative to another component) and/or a movement of the component. For illustration purposes, the coordinate system 160 may include the X-axis, the Y-axis, and the Z-axis. The X-axis and the Z-axis shown in FIG. 1 may be horizontal, and the Y-axis may be vertical. As illustrated, a positive X direction along the X-axis may be from the left side to the right side of a scanning table viewed from the direction facing the front of the medical device 110; a positive Y direction along the Y-axis may be from the lower part (or from the floor where the medical device 110 stands) to the upper part of a gantry of the medical device 110; and a positive Z direction along the Z-axis may be the direction in which the scanning table is moved from the outside into the medical device 110 viewed from the direction facing the front of the medical device 110.

This description is intended to be illustrative, and not to limit the scope of the present disclosure. Many alternatives, modifications, and variations will be apparent to those skilled in the art. The features, structures, methods, and other characteristics of the exemplary embodiments described herein may be combined in various ways to obtain additional and/or alternative exemplary embodiments. However, those variations and modifications do not depart the scope of the present disclosure. In some embodiments, the medical system 100 may include one or more additional components and/or one or more components of the medical system 100 described above may be omitted. Additionally or alternatively, two or more components of the medical system 100 may be integrated into a single component. A component of the medical system 100 may be implemented on two or more sub-components. In some embodiments, the medical system 100 may include an image archiving and communication system. In some embodiments, the image archiving and communication system may store image data digitally via an interface. The interface may include a digital imaging and communications in medicine (DICOM), a network interface, or the like. The DICOM refers to a standard for image data storage and transfer. The DICOM may use a specific file format and a communication protocol to define a medical image format that can be used for data exchange with a quality that meets clinical needs. In some embodiments, the image archiving and communication system may store a prior information database. In some embodiments, image data may be retrieved from the image archiving and communication system quickly after authorization. In some embodiments, the image archiving and communication system may have an auxiliary diagnosis management function. In some embodiments, the image archiving and communication system may transmit, manage, and store data obtained from a medical device (e.g., the medical device 110).

FIG. 2 is a schematic diagram illustrating exemplary hardware and/or software components of an exemplary computing device on which the processing device 120 may be implemented according to some embodiments of the present disclosure. As illustrated in FIG. 2, a computing device 200 may include a processor 210, storage 220, an input/output (I/O) 230, and a communication port 240.

The processor 210 may execute computer instructions (e.g., program code) and perform functions of the processing device 120 in accordance with techniques described herein. The computer instructions may include, for example, routines, programs, objects, components, data structures, procedures, modules, and functions, which perform particular functions described herein. For example, the processor 210 may process image data obtained from the medical device 110, the terminal device 140, the storage device 130, and/or any other component of the medical system 100. In some embodiments, the processor 210 may include one or more hardware processors, such as a microcontroller, a microprocessor, a reduced instruction set computer (RISC), an application specific integrated circuits (ASICs), an application-specific instruction-set processor (ASIP), a central processing unit (CPU), a graphics processing unit (GPU), a physics processing unit (PPU), a microcontroller unit, a digital signal processor (DSP), a field programmable gate array (FPGA), an advanced RISC machine (ARM), a programmable logic device (PLD), any circuit or processor capable of executing one or more functions, or the like, or any combination thereof.

Merely for illustration, only one processor is described in the computing device 200. However, it should be noted that the computing device 200 in the present disclosure may also include multiple processors. Thus operations and/or method steps that are performed by one processor as described in the present disclosure may also be jointly or separately performed by the multiple processors. For example, if in the present disclosure the processor of the computing device 200 executes both process A and process B, it should be understood that process A and process B may also be performed by two or more different processors jointly or separately in the computing device 200 (e.g., a first processor executes process A and a second processor executes process B, or the first and second processors jointly execute processes A and B).

The storage 220 may store data/information obtained from the medical device 110, the terminal device 140, the storage device 130, and/or any other component of the medical system 100. The storage 220 may be similar to the storage device 130 described in connection with FIG. 1, and the detailed descriptions are not repeated here.

The I/O 230 may input and/or output signals, data, information, etc. In some embodiments, the I/O 230 may enable a user interaction with the processing device 120. In some embodiments, the I/O 230 may include an input device and an output device. Examples of the input device may include a keyboard, a mouse, a touchscreen, a microphone, a sound recording device, or the like, or a combination thereof. Examples of the output device may include a display device, a loudspeaker, a printer, a projector, or the like, or a combination thereof. Examples of the display device may include a liquid crystal display (LCD), a light-emitting diode (LED)-based display, a flat panel display, a curved screen, a television device, a cathode ray tube (CRT), a touchscreen, or the like, or a combination thereof.

The communication port 240 may be connected to a network (e.g., the network 150) to facilitate data communications. The communication port 240 may establish connections between the processing device 120 and the medical device 110, the terminal device 140, and/or the storage device 130. The connection may be a wired connection, a wireless connection, any other communication connection that can enable data transmission and/or reception, and/or any combination of these connections. The wired connection may include, for example, an electrical cable, an optical cable, a telephone wire, or the like, or any combination thereof. The wireless connection may include, for example, a Bluetooth™ link, a Wi-Fi™ link, a WiMax™ link, a WLAN link, a ZigBee link, a mobile network link (e.g., 3G, 4G, 5G), or the like, or any combination thereof. In some embodiments, the communication port 240 may be and/or include a standardized communication port, such as RS232, RS485. In some embodiments, the communication port 240 may be a specially designed communication port. For example, the communication port 240 may be designed in accordance with the digital imaging and communications in medicine (DICOM) protocol.

FIG. 3 is a schematic diagram illustrating exemplary hardware and/or software components of an exemplary mobile device according to some embodiments of the present disclosure. In some embodiments, the terminal device 140 and/or the processing device 120 may be implemented on a mobile device 300, respectively.

As illustrated in FIG. 3, the mobile device 300 may include a communication platform 310, a display 320, a graphics processing unit (GPU) 330, a central processing unit (CPU) 340, an I/O 350, a memory 360, and storage 390. In some embodiments, any other suitable component, including but not limited to a system bus or a controller (not shown), may also be included in the mobile device 300.

In some embodiments, the communication platform 310 may be configured to establish a connection between the mobile device 300 and other components of the medical system 100, and enable data and/or signal to be transmitted between the mobile device 300 and other components of the medical system 100. For example, the communication platform 310 may establish a wireless connection between the mobile device 300 and the medical device 110, and/or the processing device 120. The wireless connection may include, for example, a Bluetooth™ link, a Wi-Fi™ link, a WiMax™ link, a WLAN link, a ZigBee link, a mobile network link (e.g., 3G, 4G, 5G), or the like, or any combination thereof. The communication platform 310 may also enable the data and/or signal between the mobile device 300 and other components of the medical system 100. For example, the communication platform 310 may transmit data and/or signals inputted by a user to other components of the medical system 100. The inputted data and/or signals may include a user instruction. As another example, the communication platform 310 may receive data and/or signals transmitted from the processing device 120. The received data and/or signals may include image data acquired by the medical device 110.

In some embodiments, a mobile operating system (OS) 370 (e.g., iOS™ Android™, Windows Phone™, etc.) and one or more applications (App(s)) 380 may be loaded into the memory 360 from the storage 390 in order to be executed by the CPU 340. The applications 380 may include a browser or any other suitable mobile apps for receiving and rendering information from the processing device 120. User interactions with the information stream may be achieved via the I/O 350 and provided to the processing device 120 and/or other components of the medical system 100 via the network 150.

To implement various modules, units, and their functionalities described in the present disclosure, computer hardware platforms may be used as the hardware platform(s) for one or more of the elements described herein. A computer with user interface elements may be used to implement a personal computer (PC) or another type of work station or terminal device, although a computer may also act as a server if appropriately programmed. It is believed that those skilled in the art are familiar with the structure, programming and general operation of such computer equipment and as a result the drawings should be self-explanatory.

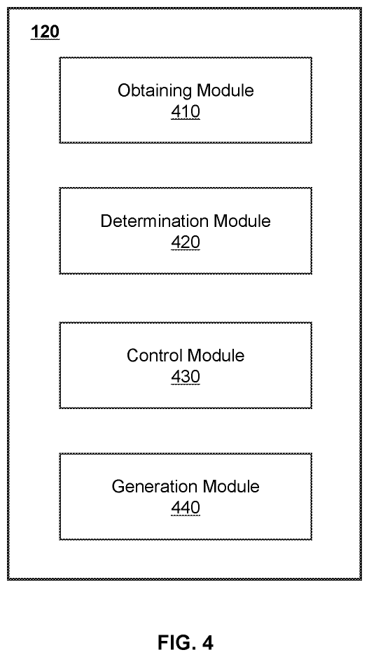

FIG. 4 is a block diagram illustrating an exemplary processing device according to some embodiments of the present disclosure. In some embodiments, the processing device 120 may include an obtaining module 410, a determination module 420, a control module 430, and a generation module 440.

The obtaining module 410 may be configured to obtain data and/or information associated with the medical system 100. The data and/or information associated with the medical system 100 may include feature information of a target subject, target prior information of a target subject, a prior information database, a value of a procedure parameter, or the like, or any combination thereof. For example, the obtaining module 410 may obtain feature information of a target subject. As another example, the obtaining module 410 may obtain target prior information of a target subject based on feature information of the target subject and a prior information database. In some embodiments, the obtaining module 410 may obtain the data and/or the information associated with the medical system 100 from one or more components (e.g., the medical device 110, the storage device 130, the terminal 140) of the medical system 100 via the network 150.

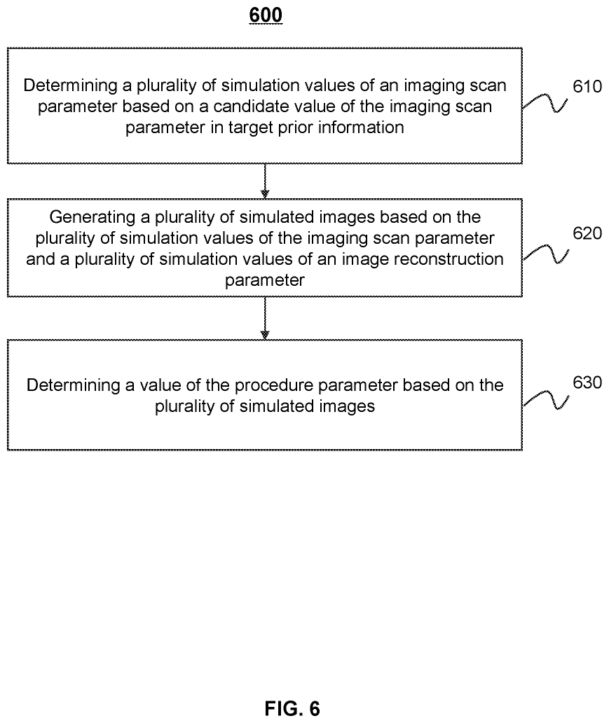

The determination module 420 may be configured to determine data and/or information associated with the medical system 100. In some embodiments, the determination module 420 may determine, based on target prior information of a target subject, a value of a procedure parameter that relates to a procedure of the target subject using a medical device. For example, the determination module 420 may determine a plurality of simulation values of an imaging scan parameter based on a candidate value of the imaging scan parameter in target prior information. The determination module 420 may generate a plurality of simulated images based on the plurality of simulation values of the imaging scan parameter and a plurality of simulation values of the image reconstruction parameter. The determination module 420 may determine a value of the procedure parameter based on the plurality of simulated images. More descriptions for determining the value of the procedure parameter may be found elsewhere in the present disclosure (e.g., FIG. 6, and descriptions thereof).

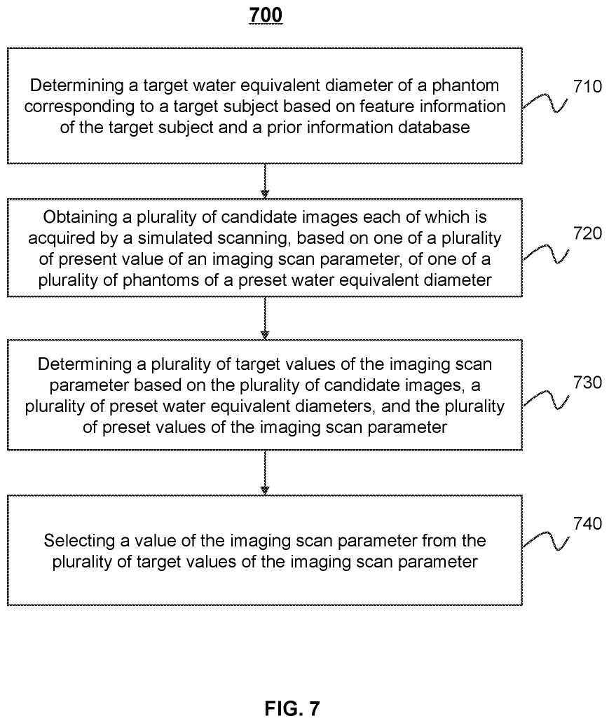

As another example, the determination module 420 may determine a target water equivalent diameter of a phantom corresponding to a target subject based on feature information of the target subject and a prior information database. The determination module 420 may obtain a plurality of candidate images. Each candidate image may be acquired by a simulated scanning, based on one of a plurality of present values of an imaging scan parameter, of one of a plurality of phantoms of a preset water equivalent diameter. The determination module 420 may determine a plurality of target values of the imaging scan parameter based on the plurality of candidate images, the plurality of preset water equivalent diameters, and the plurality of preset values of the imaging scan parameter. The determination module 420 may select a value of the imaging scan parameter from the plurality of target values of the imaging scan parameter. More descriptions for determining the value of the procedure parameter may be found elsewhere in the present disclosure (e.g., FIG. 7, and descriptions thereof).

The control module 430 may be configured to control one or more components (e.g., the medical device 110) of the medical system 100. For example, the control module 430 may cause a medical device (e.g., the medical device 110) to scan a target subject based on a value of an imaging scan parameter.

The generation module 440 may be configured to generate an image of a target subject. In some embodiments, the generation module 440 may generate an image of a target subject based on scan data of the target subject and a value of an image reconstruction parameter.

It should be noted that the above description of the processing device 120 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more modules may be combined into a single module. For example, the obtaining module 410 and the determination module 420 may be combined into a single module. In some embodiments, one or more modules may be added or omitted in the processing device 120. For example, the processing device 120 may further include a storage module (not shown in FIG. 4) configured to store data and/or information (e.g., feature information of a target subject, target prior information of a target subject, a value of a procedure parameter of a target subject, scan data of a target subject) associated with the medical system 100.

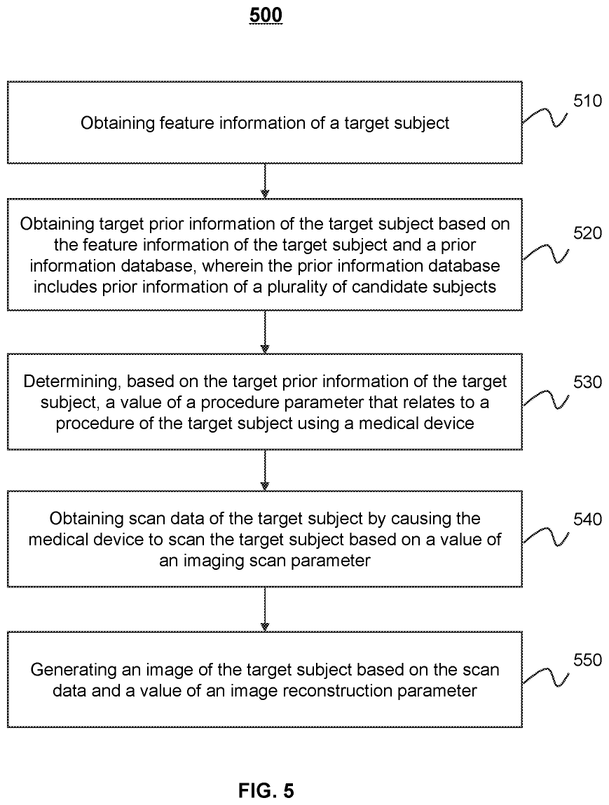

FIG. 5 is a flowchart illustrating an exemplary process for imaging according to some embodiments of the present disclosure. In some embodiments, process 500 may be executed by the medical system 100. For example, the process 500 may be implemented as a set of instructions (e.g., an application) stored in a storage device (e.g., the storage device 130, the storage device 220, and/or the storage 390). In some embodiments, the processing device 120 (e.g., the processor 210 of the computing device 200, the CPU 340 of the mobile device 300, and/or one or more modules illustrated in FIG. 4) may execute the set of instructions and may accordingly be directed to perform the process 500. The operations of the illustrated process presented below are intended to be illustrative. In some embodiments, the process 500 may be accomplished with one or more additional operations not described and/or without one or more of the operations discussed. Additionally, the order of the operations of process 500 illustrated in FIG. 5 and described below is not intended to be limiting.

In 510, the processing device 120 (e.g., the obtaining module 410) may obtain feature information of a target subject.

As used herein, a target subject refers to a subject to be scanned by a medical device (e.g., the medical device 110). For example, the target subject may be a patient to be scanned using the medical device. The feature information of the target subject may include identify information (e.g., an identification (ID) number, a name, the gender, the age, a date of birth, an occupation), contact information (e.g., a mobile phone number), medical information (e.g., a medical record number, a registration card number, a health condition, a medical history), shape information (e.g., a width, a thickness, a height, a weight) of the target subject or a portion thereof, or the like, or any combination thereof. As used herein, a width of a target subject refers to a length of the target subject (e.g., a length at the center of the target subject, a maximum length of the target subject) along a direction perpendicular to a sagittal plane of the target subject. A height of a target subject refers to a length of the target subject (e.g., a length at the center of the target subject, a maximum length of the target subject) along a direction perpendicular to a transverse plane of the target subject. A thickness of a target subject refers to a length of the target subject (e.g., a length at the center of the target subject, a maximum length of the target subject) along a direction perpendicular to a coronal plane of the target subject.

In some embodiments, the feature information (e.g., the identify information, the medical information, the shape information) of the target subject may be previously determined and stored in a storage device (e.g., the storage device 130, the storage device 220, the storage 390, or an external source). The processing device 120 may retrieve the feature information of the target subject from the storage device. Additionally or alternatively, the feature information (e.g., the shape information) of the target subject may be determined based on image data of the target subject. For example, an image capturing device (e.g., a camera) may capture the image data of the target subject, and the processing device 120 may determine the feature information of the subject based on the image data according to an image analysis algorithm (e.g., an image segmentation algorithm, a feature point extraction algorithm).

In 520, the processing device 120 (e.g., the obtaining module 410) may obtain target prior information of the target subject based on the feature information of the target subject and a prior information database. The prior information database may include prior information of a plurality of candidate subjects.

As used herein, a candidate subject refers to a subject whose data is used for establishing a prior information database. In some embodiments, the plurality of candidate subjects may include the target subject.

The prior information database may be established based on historical data of the plurality of candidate subjects, simulation data of the plurality of candidate subjects, or the like, or any combination thereof. For example, the prior information database may include historical feature information of the candidate subject, a historical scan protocol of the candidate subject, a historical value of a procedure parameter (e.g., an imaging scan parameter, an image reconstruction parameter) of the candidate subject, historical scan data (e.g., CT scan data, MRI scan data, PET scan data) of the candidate subject, a historical image of the candidate subject, a simulation value of the procedure parameter (e.g., the imaging scan parameter, the image reconstruction parameter) of the candidate subject, simulation scan data of the candidate subject, a simulated image of the candidate subject, a recommended value of the procedure parameter of the candidate subject, a dimension of a phantom (e.g., a water equivalent diameter of the phantom) corresponding to the candidate subject, a topogram image of the candidate subject, or the like, or any combination thereof. In some embodiments, the simulation scan data of the candidate subject may be determined based on the historical scan data of the candidate subject according to a low-dose simulation algorithm as described elsewhere in the present disclosure (e.g., FIG. 6 and descriptions thereof). The simulated image of the candidate subject may be generated based on the simulation scan data.

In some embodiments, the prior information database may be previously generated and stored in a storage device (e.g., the storage device 130, the storage device 220, and/or the storage 390, an external source). In some embodiments, the prior information database may be updated from time to time, e.g., periodically or not. In some embodiments, the prior information database may be updated based on data of the target subject that are at least partially different from original data from which an original prior information database is generated.

In some embodiments, the processing device 120 may obtain the target prior information of the target subject based on the feature information of the target subject. In some embodiments, the target prior information may include data (e.g., historical data, simulation data) associated with the target subject stored in the prior information database. For example, a patient may register his/her name or ID number before a scan, and target prior information of the patient may be obtained from the prior information database based on the name or the ID number of the patient.

In some embodiments, the target prior information may include data (e.g., historical data, simulation data) associated with a specific candidate subject that is similar to the target subject. In some embodiments, a degree of similarity between the specific candidate subject and the target subject may reach or exceed a threshold (e.g., 80%, 85%, 90%, 95%). The degree of similarity between the specific candidate subject and the target subject may be determined based on the feature information of the specific candidate subject and the feature information of the target subject. Merely by way of example, the processing device 120 may select a candidate subject with the highest degree of similarity to the target subject among the plurality of candidate subjects, or a portion thereof (e.g., among the plurality of candidate subjects, those who share at least one similar feature with the target subject), in the prior information database. Exemplary features may include age, gender, medical history, or the like, or any combination thereof. The processing device 120 may further designate prior information of the selected candidate subject as the target prior information of the target subject. As another example, the processing device 120 may modify at least part of the prior information of the selected candidate subject based on the feature information of the selected candidate subject and the feature information of the target subject, for example, a body shape difference between the candidate subject and the target subject. The processing device 120 may further designate the modified prior information as the target prior information of the target subject.

In 530, the processing device 120 (e.g., the determination module 420) may determine, based on the target prior information of the target subject, a value of a procedure parameter that relates to a procedure of the target subject using a medical device.

In some embodiments, the procedure may include a CT scan, an X-ray scan, or the like, to be performed by the medical device (e.g., the medical device 110). In some embodiments, the procedure parameter may include an imaging scan parameter, an image reconstruction parameter, or the like. As used herein, an imaging scan parameter refers to a parameter used for performing a scan on a target subject. For example, the imaging scan parameter may include a modality of a medical device, a voltage of a radiation source (e.g., a tube voltage), a current of the radiation source (e.g., a tube current), a distance between the radiation source and a detector (also referred to as a source image distance, or a SID), a scan time, a field of view (FOV), a scan mode, a table moving speed, a gantry rotation speed, a scan region of the target subject, a scan condition, a scan protocol, or the like, or any combination thereof. As used herein, a scan region refers to a region of the target subject to be scanned by a medical device. As used herein, a scan condition refers to a requirement of a scan process and/or a requirement of a scan result (e.g., scan data). For example, the scan condition may include a quality requirement of an image reconstructed based on the scan data. In some embodiments, value(s) of the imaging scan parameter(s) may relate to a radiation dose. In some embodiments, the radiation dose may indicate the amount of radiation per unit area to be delivered to the target subject. In some embodiments, the radiation dose may indicate a total amount of the radiation to be delivered to the target subject. In some embodiments, the radiation dose may be determined based on the tube voltage, the tube current, the scan time, or the like. For example, the value of the tube voltage may be a peak value of the tube voltage. The value of the tube voltage may be associated with an energy level or a penetration ability of X-rays, which may affect a radiation dose of a scan, a signal-to-noise ratio of a reconstructed image, and a contrast of the reconstructed image. In addition, the attenuation of tissue of the target subject may depend on the value of the tube voltage, and the value of the tube voltage may affect a CT value of the reconstructed image. As another example, the tube current may be associated with the amount of electrons emitted by a filament, that is, the amount of X-rays. In some embodiments, the value of the tube voltage may be expressed as kV, and the value of the tube current may be expressed as mA. In some embodiments, mAs may be defined as the product of the value of the tube current and an exposure time. The mAs may have a linear relationship with the radiation dose. For example, a relatively low value of mAs may correspond to a relatively low radiation dose.

As used herein, an image reconstruction parameter refers to a parameter used for reconstructing an image of a target subject based on scan data of the target subject. For example, the image reconstruction parameters may include parameters of a reconstruction algorithm, an image quality evaluation parameter (e.g., a density resolution, a spatial resolution, a signal-to-noise ratio), or the like, or any combination thereof.

In some embodiments, a first modality of the medical device may be the same as a second modality corresponding to the target prior information. For example, a first modality of a first medical device that is to be used to scan the target subject may be the same as a second modality of a second medical device that was used to generate the target prior information. The first modality or the second modality may include an ultrasound imaging, an X-ray imaging, a computed tomography (CT), a magnetic resonance imaging (MRI), a single photon emission computed tomography (SPECT), a positron emission tomography (PET), or the like. For illustration purposes, the first medical device and the second medical device may both be a CT device. In this situation, the processing device 120 may determine the value of the procedure parameter based on the target prior information of the target subject.

In some embodiments, the target prior information may include a candidate value (e.g., a historical value, a simulation value) of the procedure parameter of the target subject. In some embodiments, the processing device 120 may designate the candidate value of the procedure parameter in the target prior information as the value of the procedure parameter. In some embodiments, at least part of the feature information of the target subject may be different from historical feature information of the target subject in the target prior information. For example, an actual body shape of the target subject may be different from a historical body shape of the target subject in the target prior information. In some embodiments, a scan condition of the target subject may be different form a historical scan condition of the target subject in the target prior information. For example, an actual scan region of the target subject may be different from a historical scan region of the target subject in the target prior information. As another example, an actual quality requirement of a reconstructed image of the target subject may be different from a historical quality requirement of a reconstructed image of the target subject in the target prior information. In this situation, the processing device 120 may modify at least part of the candidate value of the procedure parameter of the target subject in the target prior information based on the feature information (e.g., the age, the body shape, the scan region) and/or the scan condition of the target subject. The processing device 120 may further designate the modified candidate value of the procedure parameter as the value of the procedure parameter. For example, if a historical scan region is the feet of the target subject, and an actual scan region is the head of the target subject, the processing device 120 may determine a value of the procedure parameter (e.g., a tube voltage, a tube current) corresponding to the head by increasing a historical value of the procedure parameter corresponding to the feet, due to the head have more osseous tissue than the feet. As another example, if a historical scan region is the feet of the target subject, and an actual scan region is the lungs of the target subject, the processing device 120 may determine a value of the procedure parameter (e.g., a tube voltage, a tube current) corresponding to the lungs by decreasing a historical value of the procedure parameter corresponding to the feet, due to the feet have more osseous tissue than the lungs.

In some embodiments, the processing device 120 may determine the value of the procedure parameter based on the target prior information and a scan condition of the target subject. For example, the processing device 120 may determine whether the candidate value (e.g., a historical value, a simulation value) of the procedure parameter of the target subject in the target prior information satisfies the scan condition. In response to determining that the candidate value of the procedure parameter satisfies the scan condition, the processing device 120 may determine the candidate value of the procedure parameter as the value of the procedure parameter. In response to determining that the candidate value of the procedure parameter does not satisfy the scan condition, the processing device 120 may adjust (by, e.g., increasing, decreasing) the candidate value of the procedure parameter. The processing device 120 may determine the adjusted candidate value of the procedure parameter as the value of the procedure parameter.

In some embodiments, the target prior information may include a candidate value of the imaging scan parameter of the target subject. The processing device 120 may determine a plurality of simulation values of the imaging scan parameter based on the candidate value of the imaging scan parameter. The processing device 120 may generate a plurality of simulated images based on the plurality of simulation values of the imaging scan parameter and a plurality of simulation values of the image reconstruction parameter. The processing device 120 may determine the value of the procedure parameter based on the plurality of simulated images. More descriptions for determining the value of the procedure parameter may be found elsewhere in the present disclosure (e.g., FIG. 6 and descriptions thereof).

In some embodiments, the first modality of the medical device may be different from the second modality corresponding to the target prior information. For example, the first modality of the first medical device that is to be used to scan the target subject may be different from the second modality of the second medical device that was used to generate the target prior information. For illustration purposes, the first medical device may be a CT device, and the second medical device may be an MRI device. In this situation, the processing device 120 may process the target prior information, and determine the value of the procedure parameter based on the processed target prior information. For example, the processing device 120 may generate a first image of the first modality based on a second image of the second modality in the target prior information. The processing device 120 may determine the value of the procedure parameter based on the second image. More descriptions for determining the value of the procedure parameter may be found elsewhere in the present disclosure (e.g., FIG. 6 and descriptions thereof).

In some embodiments, the processing device 120 may determine a value of the imaging scan parameter (e.g., a tube voltage, a tube current) based on the feature information (e.g., a body shape) of the target subject and a scan condition of the target subject according to an auto-Kv technology. For example, the processing device 120 may determine a dimension (e.g., a target water equivalent diameter) of a phantom corresponding to the target subject based on the feature information of the target subject and the prior information database. The processing device 120 may determine the value of the imaging scan parameter based on the dimension (e.g., the target water equivalent diameter) of the phantom corresponding to the target subject. More descriptions for determining the value of the imaging scan parameter based on the dimension of the phantom may be found elsewhere in the present disclosure (e.g., FIG. 7 and descriptions thereof).

In 540, the processing device 120 (e.g., the control module 430) may obtain scan data of the target subject by causing the medical device to scan the target subject based on a value of the imaging scan parameter.

In some embodiments, the processing device 120 may determine position(s) of component(s) (e.g., a collimator, a radiation source, a scanning table, a detector) of the medical device (e.g., the medical device 110) based on value(s) of the imaging scan parameter(s) (e.g., a distance between a radiation source and a detector). After the component(s) of the medical device are located at their respective position(s), the processing device 120 may control the medical device (e.g., the medical device 110) to scan the target subject based on the value(s) of the imaging scan parameter(s) (e.g., a current of the radiation source, a voltage of a radiation source). In some embodiments, the processing device 120 may obtain the scan data from the medical device (e.g., the medical device 110). In some embodiments, the scan data may be stored in a storage device (e.g., the storage device 130, the storage device 220, the storage 390, or an external source). The processing device 120 may obtain the scan data of the target subject from the storage device.

In 550, the processing device 120 (e.g., the generation module 440) may generate an image of the target subject based on the scan data and a value of the image reconstruction parameter. In some embodiments, the processing device 120 may generate the image of the target subject based on the scan data and value(s) of the image reconstruction parameter(s).

In some embodiments, after the scan is performed on the target subject, the feature information of the target subject, the scan data of the target subject, the value of the procedure parameter of the target subject may be stored in the prior information database to update the prior information database.

Traditionally, a value of the procedure parameter of the target subject may be a default value set by a manufacturer of a medical device, or an experimental value obtained based on a phantom experiment, which may be unsuitable for patients of different body shapes and/or different clinical scenarios. To ensure a successful scan in which imaging data of sufficient quality is generated, the dose of the imaging medium (e.g., radiation) may be increased and/or scanning time may be extended, thereby causing unnecessary exposure to excessive imaging medium and/or discomfort from the extended scanning time. According to some embodiments of the present disclosure, the value of the procedure parameter (e.g., the value of the imaging scan parameter, the value of the image reconstruction parameter) of the target subject may be determined based on the target prior information of the target subject obtained from the prior information database, which may improve the efficiency, accuracy, and/or reliability of the parameter determination process. Moreover, the process may be automated, thereby reducing user involvement and/or inter-user variability in the parameter determination. In addition, the target subject may be scanned based on the value of the imaging scan parameter, and the image of the target subject may be generated based on the value of the image reconstruction parameter, which may reduce a radiation dose while maintaining a desired image quality acquired based on a scan.

It should be noted that the above description regarding the process 500 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the processing device 120 may determine values of a plurality of procedure parameters (e.g., a plurality of imaging scan parameters, a plurality of image reconstruction parameters) based on the target prior information of the target subject according to process 500. For example, the processing device 120 may determine values of a plurality of imaging scan parameters (e.g., the tube voltage, the tube current, the scan time) related to the radiation dose of the target subject according to process 500. As another example, the processing device 120 may determine values of a plurality of image reconstruction parameters related to an image reconstruction algorithm according to process 500.

FIG. 6 is a flowchart illustrating an exemplary process for determining a value of a procedure parameter according to some embodiments of the present disclosure. In some embodiments, process 600 may be executed by the medical system 100. For example, the process 600 may be implemented as a set of instructions (e.g., an application) stored in a storage device (e.g., the storage device 130, the storage device 220, and/or the storage 390). In some embodiments, the processing device 120 (e.g., the processor 210 of the computing device 200, the CPU 340 of the mobile device 300, and/or one or more modules illustrated in FIG. 4) may execute the set of instructions and may accordingly be directed to perform the process 600. The operations of the illustrated process presented below are intended to be illustrative. In some embodiments, the process 600 may be accomplished with one or more additional operations not described and/or without one or more of the operations discussed. Additionally, the order of the operations of process 600 illustrated in FIG. 6 and described below is not intended to be limiting. In some embodiments, operation 530 in FIG. 5 may be performed according to process 600.

In 610, the processing device 120 (e.g., the determination module 420) may determine a plurality of simulation values of an imaging scan parameter based on a candidate value of the imaging scan parameter in target prior information.

In some embodiments, the processing device 120 may determine the plurality of simulation values of the imaging scan parameter based on the candidate value of the imaging scan parameter and a plurality of dose reduction ratios. The plurality of dose reduction ratios may be determined manually by a user (e.g., a doctor) of the medical system 100 or by one or more components (e.g., the processing device 120) of the medical system 100 according to different situations. For example, the plurality of dose reduction ratios may include 5%, 10%, 15%, 20%, 25%, or the like. For illustration purposes, if a candidate value of the tube voltage is 150 kV, and a dose reduction ratio is 10%, the processing device 120 may determine that a simulation value of the tube voltage is 135 kV (150−150*10%=135).

In some embodiments, the processing device 120 may obtain an initial value of the imaging scan parameter. In some embodiments, the initial value of the imaging scan parameter may be an expected value of the imaging scan parameter. The initial value of the imaging scan parameter may be determined manually by a user (e.g., a doctor) of the medical system 100 or by one or more components (e.g., the processing device 120) of the medical system 100 according to different situations. The processing device 120 may obtain an initial image based on the initial value of the imaging scan parameter and the candidate value of the imaging scan parameter using a dose simulation model. The dose simulation model refers to a model (e.g., a machine learning model) or an algorithm for determining a first image corresponding to a first value of the imaging scan parameter based on a second image corresponding to a second value of the imaging scan parameter. The first value of the imaging scan parameter may be different from the second value of the imaging scan parameter. For example, the first value of imaging scan parameter may be lower than the second value of the imaging scan parameter. For example, the processing device 120 may input the initial value of the imaging scan parameter, the candidate value of the imaging scan parameter, and an image corresponding to the candidate value of the imaging scan parameter (i.e., an image reconstructed based on candidate scan data obtained based on the candidate value of the imaging scan parameter) into the dose simulation model, and the dose simulation model may output the initial image corresponding to the initial value of the imaging scan parameter.

In some embodiments, the dose simulation model may be obtained by training a preliminary model using a plurality of training samples. In some embodiments, the dose simulation model may be predetermined by a computing device (e.g., the processing device 120 or a computing device of a vendor of the dose simulation model) and stored in a storage device (e.g., the storage device 130, the storage 220, the storage 390, or an external source). The processing device 120 may obtain the dose simulation model from the storage device. Alternatively, the processing device 120 may determine the dose simulation model by performing a training.

To train the dose simulation model, a plurality of training samples may be used. Each training sample may include a sample value of the imaging scan parameter, a sample image corresponding to the sample value of the imaging scan parameter, a reference value of the imaging scan parameter, and a reference image (also referred to as a gold standard image) corresponding to the reference value of the imaging scan parameter. In some embodiments, the reference value of the imaging scan parameter may be different from the sample value of the imaging scan parameter. For example, the reference value of the imaging scan parameter may be lower than the sample value of the imaging scan parameter. In some embodiments, the sample image and the reference image may be historical images reconstructed based on historical scan data of a sample subject during a historical scan. The sample subject may be the same as or different from the target subject. In some embodiments, the sample image may be a historical image reconstructed based on the historical scan data of the sample during the historical scan. The reference image may be obtained based on the sample image, the sample value of the imaging scan parameter, and the reference value of the imaging scan parameter, using one or more existing low-dose simulation algorithms.

In some embodiments, the preliminary model may be of any type of machine learning model. Merely by way of example, the preliminary model may include an artificial neural network (ANN), a random forest model, a support vector machine, a decision tree, a convolutional neural network (CNN), a recurrent neural network (RNN), a deep learning model, a Bayesian network, a K-nearest neighbor (KNN) model, a generative adversarial network (GAN) model, etc. The training of the preliminary model may be implemented according to a machine learning algorithm, such as an artificial neural network algorithm, a deep learning algorithm, a decision tree algorithm, an association rule algorithm, an inductive logic programming algorithm, a support vector machine algorithm, a clustering algorithm, a Bayesian network algorithm, a reinforcement learning algorithm, a representation learning algorithm, a similarity and metric learning algorithm, a sparse dictionary learning algorithm, a genetic algorithm, a rule-based machine learning algorithm, or the like, or any combination thereof. The machine learning algorithm used to generate the dose simulation model may be a supervised learning algorithm, a semi-supervised learning algorithm, an unsupervised learning algorithm, or the like.

In some embodiments, the dose simulation model may be determined by performing a plurality of iterations to iteratively update one or more parameter values of the preliminary model. For each of the plurality of iterations, a specific training sample may first be input into the preliminary model. For example, a sample value of the imaging scan parameter, a sample image corresponding to the sample value of the imaging scan parameter, and a reference value of the imaging scan parameter in the specific training sample may be inputted into an input layer of the preliminary model, and a reference image corresponding to the reference value of the imaging scan parameter in the specific training sample may be inputted into an output layer of the preliminary model as a desired output of the preliminary model. The preliminary model may add simulation noises on the sample image based on the sample value of the imaging scan parameter and the reference value of the imaging scan parameter, to determine a predicted output (i.e., a predicted image corresponding to the reference value of the imaging scan parameter) of the specific training sample. The predicted output (i.e., the predicted image) may then be compared with the desired output (e.g., the reference image) based on a cost function. As used herein, a cost function of a machine learning model may be configured to assess a difference between a predicted output (e.g., the predicted image) of the machine learning model and a desired output (e.g., the reference image). If the value of the cost function exceeds a threshold in a current iteration, parameter values of the preliminary model may be adjusted and/or updated in order to decrease the value of the cost function (i.e., the difference between the predicted image and the reference image) to smaller than the threshold, and an intermediate model may be generated. Accordingly, in the next iteration, another training sample may be input into the intermediate model to train the intermediate model as described above.

The plurality of iterations may be performed to update the parameter values of the preliminary model (or the intermediate model) until a termination condition is satisfied. The termination condition may provide an indication of whether the preliminary model (or the intermediate model) is sufficiently trained. The termination condition may relate to the cost function or an iteration count of the iterative process or training process. For example, the termination condition may be satisfied if the value of the cost function associated with the preliminary model (or the intermediate model) is minimal or smaller than a threshold (e.g., a constant). As another example, the termination condition may be satisfied if the value of the cost function converges. The convergence may be deemed to have occurred if the variation of the values of the cost function in two or more consecutive iterations is smaller than a threshold (e.g., a constant). As still another example, the termination condition may be satisfied when a specified number (or count) of iterations are performed in the training process. The dose simulation model may be determined based on the updated parameter values.

It should be noted that the above description is merely provided for the purposes of illustration, and not intended to limit the scope of the present disclosure. In some embodiments, the training sample may further include feature information (e.g., a density resolution, a spatial resolution, a signal-to-noise ratio) of the sample image corresponding to the sample value of the imaging scan parameter and/or feature information (e.g., a density resolution, a spatial resolution, a signal-to-noise ratio, a size) of the reference image corresponding to the reference value of the imaging scan parameter.

In some embodiments, the dose simulation model may be updated from time to time, e.g., periodically or not, based on a sample set that is at least partially different from an original sample set from which an original dose simulation model is determined. For instance, the dose simulation model may be updated based on a sample set including new samples that are not in the original sample set, samples processed using an intermediate model of a prior version, or the like, or a combination thereof. In some embodiments, the determination and/or updating of the dose simulation model may be performed on a processing device, while the application of the dose simulation model may be performed on a different processing device. In some embodiments, the determination and/or updating of the dose simulation model may be performed on a processing device of a system different than the medical system 100 or a server different than a server including the processing device 120 on which the application of the dose simulation model is performed. For instance, the determination and/or updating of the dose simulation model may be performed on a first system of a vendor who provides and/or maintains such a dose simulation model and/or has access to training samples used to determine and/or update the dose simulation model, while parameter determination based on the provided dose simulation model may be performed on a second system of a client of the vendor. In some embodiments, the determination and/or updating of the dose simulation model may be performed online in response to a request for parameter determination. In some embodiments, the determination and/or updating of the dose simulation model may be performed offline.

Further, the processing device 120 may determine at least one of the plurality of simulation values of the imaging scan parameter based on the initial image and the initial value of the imaging scan parameter. In some embodiments, the processing device 120 may determine whether the quality of the initial image satisfies an image quality evaluation requirement. For example, the processing device 120 may determine whether the quality (e.g., a density resolution, a spatial resolution, a signal-to-noise ratio) of the initial image is higher than a quality threshold (e.g., a density resolution threshold, a spatial resolution threshold, a signal-to-noise ratio threshold). In response to determining that the quality of the initial image is higher than the quality threshold, the processing device 120 may determine that the quality of the initial image satisfies the image quality evaluation requirement. The processing device 120 may determine the initial value of the imaging scan parameter as the simulation value of the imaging scan parameter. In response to determining that the quality of the initial image does not satisfy the image quality evaluation requirement, the processing device 120 may adjust (e.g., increase) the initial value of the imaging scan parameter, and determine the adjusted initial value of the imaging scan parameter as the simulation value of the imaging scan parameter.

According to some embodiments of the present disclosure, the plurality of simulation values of the imaging scan parameter may be determined using the dose simulation model. The dose simulation model may be generated based on deep learning. With the dose simulation model obtained based on the deep learning, the parameter determination process may be simplified, and accordingly the efficiency and the accuracy of the parameter determination process may be improved.

In 620, the processing device 120 (e.g., the determination module 420) may generate a plurality of simulated images based on the plurality of simulation values of the imaging scan parameter and a plurality of simulation values of the image reconstruction parameter.

In some embodiments, the target prior information may include candidate scan data corresponding to the candidate value of the imaging scan parameter of the target subject. As used herein, “scan data corresponding to a value of an imaging scan parameter (e.g., the candidate scan data corresponding to the candidate value of the imaging scan parameter)” refers to scan data obtained by performing a scan on a subject using a medical device according to the value of the imaging scan parameter.

In some embodiments, for each simulation value of the plurality of simulation values of the imaging scan parameter, the processing device 120 may determine simulation scan data corresponding to the simulation value of the imaging scan parameter based on the candidate scan data corresponding to the candidate value of the imaging scan parameter, the candidate value of the imaging scan parameter, and the simulation value of the imaging scan parameter. For example, the processing device 120 may determine the simulation scan data based on the candidate scan data, the candidate value of the imaging scan parameter, and the simulation value of the imaging scan parameter, according to a low-dose simulation algorithm. The low-dose simulation algorithm may be used to generate first scan data (e.g., low-dose CT data) corresponding to a relatively low radiation dose (e.g., a relatively small value of the imaging scan parameter) based on second scan data (e.g., high-dose CT data) corresponding to a relatively high radiation dose (e.g., a relatively large value of the imaging scan parameter) according to a statistical correlation between the first scan data corresponding to the relatively low radiation dose and the second scan data corresponding to the relatively high radiation dose, while ensuring the quality of an image reconstructed based on the first scan data corresponding to the relatively low radiation dose.

In some embodiments, the processing device 120 may determine the simulation scan data by adding noise in the candidate scan data based on the candidate value of the imaging scan parameter, the simulation value of the imaging scan parameter, and a statistical distribution function of the noise. For example, the noise in CT scan data usually comes from a photon noise and an electronic noise. The photon noise may be associated with a radiation dose and feature information of the target subject. The probability density function of the photon noise may satisfy the Poisson distribution. That is, the statistical distribution function of the photon noise may satisfy the Poisson distribution. The electronic noise may be associated with a system hardware (e.g., a readout circuit of a detector). The probability density function of the electronic noise may satisfy the Gaussian distribution. That is, the statistical distribution function of the electronic noise may satisfy the Gaussian distribution. Merely by way of example, the processing device 120 may determine simulation CT scan data based on a candidate value of a tube current (e.g., a historical value of the tube current), candidate CT scan data (e.g., historical CT scan data), a simulation value of the tube current, and a statistical distribution function of the noise according to Equation (1):

s_{β}=s_{α}(β−α/α)^{1/2}G(0,s_{α}) (1),

where s_{β} refers to simulation CT scan raw data; s_{α} refers to candidate CT raw data (e.g., historical CT raw data); refers to a simulation value of the tube current; a refers to a candidate value of the tube current (e.g., a historical value of the tube current); G refers to a random number generator that satisfies the Gaussian distribution, the mean of the Gaussian distribution is 0, and the variance of the Gaussian distribution is sₐ.

Further, for each simulation value of the plurality of simulation values of the image reconstruction parameter, the processing device 120 may generate a simulated image based on the simulation scan data and the simulation value of the image reconstruction parameter. For example, the processing device 120 may generate a plurality of simulated images based on different parameter values of a same image reconstruction algorithm. As another example, the processing device 120 may generate a plurality of simulated images based on different parameter values of different image reconstruction algorithms.

In 630, the processing device 120 (e.g., the determination module 420) may determine a value of the procedure parameter based on the plurality of simulated images.

In some embodiments, the processing device 120 may determine the value of the procedure parameter based on the quality of the plurality of simulated images. For example, the processing device 120 may select a simulated image with the highest quality (e.g., the highest density resolution, the highest spatial resolution, the highest signal-to-noise ratio) from the plurality of simulated images. The processing device 120 may determine the simulation value of the procedure parameter corresponding to the selected simulated image as the value of the procedure parameter of the target subject. In some embodiments, the processing device 120 may adjust the simulation value of the procedure parameter corresponding to the selected simulated image, and determine the adjusted simulation value of the procedure parameter as the value of the procedure parameter of the target subject.

As another example, the processing device 120 may select a plurality of candidate simulated images from the plurality of simulated images based on a quality threshold (e.g., a density resolution threshold, a spatial resolution threshold, a signal-to-noise ratio threshold). The quality of the plurality of candidate simulated images may be equal to or higher than the quality threshold. The processing device 120 may select a target simulated image from the plurality of candidate simulated images. The simulation value of the imaging scan parameter corresponding to the target simulated image may be lower than other values of the imaging scan parameter corresponding to the other candidate simulated images. The processing device 120 may determine the simulation value of the procedure parameter corresponding to the target simulated image as the value of the procedure parameter of the target subject. In some embodiments, the processing device 120 may adjust the simulation value of the procedure parameter corresponding to the target simulated image, and determine the adjusted simulation value of the procedure parameter as the value of the procedure parameter of the target subject.

According to some embodiments of the present disclosure, simulation scan data corresponding to a simulation value of the imaging scan parameter may be obtained based on candidate scan data corresponding to a candidate value of the imaging scan parameter according to a low-dose simulation algorithm. By using the low-dose simulation algorithm, the simulation scan data may be obtained efficiently and accurately, which may further improve the efficiency of image reconstruction based on the simulation scan data. A plurality of simulated images may then be generated based on the simulation scan data and a plurality of simulation values of the image reconstruction parameter associated with different reconstruction algorithms (e.g., a deep learning algorithm, an iterative reconstruction algorithm). The value of the procedure parameter may further be determined based on the quality of the plurality of simulated images, which may reduce a radiation dose while maintaining a desired image quality during a scan.

It should be noted that the above description regarding the process 600 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

In some embodiments, the target prior information may include a candidate radiation dose (e.g., a historical radiation dose) of the target subject. The processing device 120 may determine a plurality of simulation radiation doses based on the candidate radiation dose. In some embodiments, the processing device 120 may determine the plurality of simulation radiation doses based on the candidate radiation dose and a plurality of dose reduction ratios.

In some embodiments, the processing device 120 may obtain an initial radiation dose. In some embodiments, the initial radiation dose may be an expected radiation dose. The initial radiation dose may be determined manually by a user (e.g., a doctor) of the medical system 100 or by one or more components (e.g., the processing device 120) of the medical system 100 according to different situations. The processing device 120 may obtain an initial image based on the initial radiation dose and the candidate radiation dose using a second dose simulation model. The second dose simulation model refers to a model (e.g., a machine learning model) or an algorithm for determining a first image corresponding to a first radiation dose based on a second image corresponding to a second radiation dose. The first radiation dose may be different from the second radiation dose. For example, the first radiation dose may be lower than the second radiation dose. The training of the second dose simulation model may be similar to the training of the dose simulation model as described in connection with operation 610.

Further, the processing device 120 may determine at least one of the plurality of simulation radiation doses based on the initial image and the initial radiation dose. In some embodiments, the processing device 120 may determine whether the quality of the initial image satisfies an image quality evaluation requirement. For example, the processing device 120 may determine whether the quality (e.g., a density resolution, a spatial resolution, a signal-to-noise ratio) of the initial image is higher than a quality threshold (e.g., a density resolution threshold, a spatial resolution threshold, a signal-to-noise ratio threshold). In response to determining that the quality of the initial image is higher than the quality threshold, the processing device 120 may determine that the quality of the initial image satisfies the image quality evaluation requirement. The processing device 120 may determine the initial radiation dose as the simulation radiation dose. In response to determining that the quality of the initial image does not satisfy the image quality evaluation requirement, the processing device 120 may adjust (e.g., increase) the initial radiation dose, and determine the adjusted radiation dose as the simulation radiation dose.

The processing device 102 may generate a plurality of simulated images based on the plurality of simulation radiation doses. The processing device 120 may determine the value of the procedure parameter based on the plurality of simulated images. In some embodiments, the processing device 120 may determine the value of the procedure parameter based on the quality of the plurality of simulated images as described in connection with operation 630. For example, the processing device 120 may select a target simulated image with the highest quality from the plurality of simulated images. The processing device 120 may determine candidate value of the imaging scan parameter and candidate value of the image reconstruction parameter corresponding to the target simulated image as the value of the procedure parameter of the target subject.

In some embodiments, the processing device 120 may optimize a low-dose simulation algorithm based on prior information in the prior information database. For example, the prior information database may include historical data (e.g., historical scan data, a historical image) corresponding to a relatively large value of the imaging scan parameter, and historical data (e.g., historical scan data, a historical image) corresponding to a relatively small value of the imaging scan parameter. Empirical parameters of the low-dose simulation algorithm may be determined and/or adjusted based on the historical data corresponding to a relatively large value of the imaging scan parameter, and historical data corresponding to a relatively small value of the imaging scan parameter. In some embodiments, for different medical devices, the empirical parameters of the low-dose simulation algorithm may be different. Different prior information databases may be established for different medical devices. The empirical parameters of the low-dose simulation algorithm corresponding to a specific medical device may be determined and/or adjusted based on the prior information database for the specific medical device. The prior information database for the specific medical device may include historical data (e.g., historical scan data, a historical image, a historical value of the procedure parameter) generated by scanning a subject using the specific medical device.

In some embodiments, process 600 may be performed before a procedure (e.g., a CT scan, an MRI scan) of the target subject to determine the value of the procedure parameter. In some embodiments, process 600 may be performed after the procedure of the target subject to determine a recommended value of the procedure parameter. The recommended value of the procedure parameter may be used for the next procedure of the target subject. For example, after a scan is performed on the target subject based on an actual value of the imaging scan parameter, the processing device 120 may determine a plurality of simulation values of the imaging scan parameter based on the actual value of the imaging scan parameter as described in connection with operation 610. The processing device 120 may generate a plurality of simulated images based on the plurality of simulation values of the imaging scan parameter and a plurality of simulation values of the image reconstruction parameter as described in connection with operation 620. The processing device 120 may determine a recommended value of the procedure parameter based on the plurality of simulated images as described in connection with operation 630. For example, the processing device 120 may determine the recommended value of the procedure parameter based on the quality of the plurality of simulated images. The recommended value of the procedure parameter of the target subject may be stored in the prior information database.

In some embodiments, before a scan is performed on the target subject, the processing device 120 may determine whether the prior information database includes a recommended value of the procedure parameter of the target subject based on the feature information (e.g., the name, the ID number) of the target subject. In response to determining that the prior information database does not include a recommended value of the procedure parameter of the target subject, the processing device 120 may determine the value of the procedure parameter of the target subject as described in process 600. In response to determining that the prior information database includes a recommended value of the procedure parameter of the target subject, the processing device 120 may determine whether the recommended value of the procedure parameter satisfies a scan condition of the target subject. For example, the processing device 120 may determine whether a historical scan protocol corresponding to the recommended value of the procedure parameter is the same as or similar to a current scan protocol of the target subject. As another example, the processing device 120 may determine whether an image corresponding to the recommended value of the procedure parameter satisfies a quality requirement (e.g., higher than a quality threshold). In response to determining that the recommended value of the procedure parameter satisfies the scan condition, the processing device 120 may determine the value of the procedure parameter based on the recommended value of the procedure parameter. For example, the processing device 120 may determine the recommended value of the procedure parameter as the value of the procedure parameter of the target subject. As another example, the processing device 120 may adjust the recommended value of the procedure parameter based on the scan condition of the target subject and/or the feature information of the target subject. The processing device 120 may determine the adjusted recommended value of the procedure parameter as the value of the procedure parameter of the target subject.

In some embodiments, before a scan is performed on the target subject, the processing device 120 may determine whether the prior information database includes a recommended radiation dose of the target subject based on the feature information (e.g., the name, the ID number) of the target subject. In response to determining that the prior information database does not include a recommended radiation dose of the target subject, the processing device 120 may determine the value of the procedure parameter of the target subject as described in process 600. In response to determining that the prior information database includes a recommended value of the radiation dose of the target subject, the processing device 120 may determine whether the recommended radiation dose satisfies a scan condition of the target subject. For example, the processing device 120 may determine whether a historical scan protocol corresponding to the recommended radiation dose is the same as or similar to a current scan protocol of the target subject. As another example, the processing device 120 may determine whether an image corresponding to the recommended radiation dose satisfies a quality requirement (e.g., higher than a quality threshold). In response to determining that the recommended radiation dose satisfies the scan condition, the processing device 120 may determine the value of the procedure parameter based on the recommended radiation dose. For example, the processing device 120 may determine values of a plurality of imaging scan parameters (e.g., the tube voltage, the tube current, and the scan time) based on the recommended radiation dose.

According to some embodiments of the present disclosure, after a current scan is performed on the target subject, the recommended value of the procedure parameter of the target subject for the next scan may be determined based on the value of the procedure parameter of the target subject in the current scan, which may reduce the time for parameter determination in the next scan, and may further improve the efficiency of the next scan of the target subject.

In some embodiments, the first modality of the medical device may be different from the second modality corresponding to the target prior information. For example, the first modality of the first medical device that is to be used to scan the target subject may be different from the second modality of the second medical device that was used to generate the target prior information. In some embodiments, the processing device 120 may generate a first image of the first modality based on a second image of the second modality in the target prior information. For example, the processing device 120 may generate the first image of the first modality based on the second image of the second modality according to one or more modality conversion algorithms. As another example, the processing device 120 may generate the first image of the first modality based on the second image of the second modality using a modality model. The modality model may be configured to generate the first image of the first modality based on the second image of the second modality. For illustration purposes, an MR image-to-CT image translation process is taken as an example, the processing device 120 may obtain an MR image of the target subject acquired by an MRI device from the prior information database. The processing device 120 may input the MR image into the modality model. The modality model may output a CT image corresponding to the MR image. The CT image may correspond to a reconstruction algorithm (e.g., a filtered back-projection algorithm, an iterative reconstruction algorithm).

In some embodiments, the modality model may be obtained by training a preliminary model using a plurality of training samples. In some embodiments, the modality model may be predetermined by a computing device (e.g., the processing device 120 or a computing device of a vendor of the modality model) and stored in a storage device (e.g., the storage device 130, the storage 220, the storage 390, or an external source). The processing device 120 may obtain the modality model from the storage device. Alternatively, the processing device 120 may determine the modality model by performing a training.

To train the modality model, a plurality of training samples may be used. Each training sample may include a sample image, a sample value of procedure parameter corresponding to the sample image, a reference image, a reference value of the procedure parameter corresponding to the reference image. In some embodiments, the modality of the sample image may be different from the modality of the reference image. In some embodiments, the modality model may be determined by performing a plurality of iterations to iteratively update one or more parameter values of the preliminary model. The training of the modality model may be similar to the training of the dose simulation model as described elsewhere in the present disclosure.

Further, the processing device 120 may determine the value of the procedure parameter based on the second image. For example, in response to determining that the quality of the second image is equal to or higher than a quality threshold, the processing device 120 may determine a candidate value of the procedure parameter corresponding to the second image as the value of the procedure parameter of the target subject. In response to determining that the quality of the second image is lower than the quality threshold, the processing device 120 may adjust the candidate value of the procedure parameter corresponding to the second image, and determine the adjusted candidate value of the procedure parameter corresponding to the second image as the value of the procedure parameter of the target subject. As another example, the processing device 120 may obtain a plurality of simulation values of the imaging scan parameter based on a candidate value of the imaging scan parameter corresponding to the second image as described in connection with operation 610. The processing device 120 may generate a plurality of simulated images based on the plurality of simulation values of the imaging scan parameter and a plurality of simulation values of the image reconstruction parameter as described in connection with operation 620. The processing device 120 may determine a value of the procedure parameter based on the plurality of simulated images as described in connection with operation 630.

According to some embodiments of the present disclosure, the modality conversion between images of different modalities may be realized by using a deep learning neural network (i.e., the modality model), the prior information of the target subject of all modalities may be fully utilized, and the utilization rate of the prior information database may be improved.

FIG. 7 is a flowchart illustrating an exemplary process for determining a value of a procedure parameter according to some embodiments of the present disclosure. In some embodiments, process 700 may be executed by the medical system 100. For example, the process 700 may be implemented as a set of instructions (e.g., an application) stored in a storage device (e.g., the storage device 130, the storage device 220, and/or the storage 390). In some embodiments, the processing device 120 (e.g., the processor 210 of the computing device 200, the CPU 340 of the mobile device 300, and/or one or more modules illustrated in FIG. 4) may execute the set of instructions and may accordingly be directed to perform the process 700. The operations of the illustrated process presented below are intended to be illustrative. In some embodiments, the process 700 may be accomplished with one or more additional operations not described and/or without one or more of the operations discussed. Additionally, the order of the operations of process 700 illustrated in FIG. 7 and described below is not intended to be limiting. In some embodiments, operation 530 in FIG. 5 may be performed according to process 700.

In 710, the processing device 120 (e.g., the determination module 420) may determine a target water equivalent diameter of a phantom corresponding to a target subject based on feature information of the target subject and a prior information database.

As used herein, a phantom corresponding to a target subject refers to a mathematical model used to represent tissues or organs in the target subject. In some embodiments, a dimension of the phantom may include a target water equivalent diameter of the phantom.

In some embodiments, the processing device 120 may determine the target water equivalent diameter based on candidate scan data of the target subject in the prior information database. The candidate scan data may include CT scan data, MRI scan data, PET scan data, or the like, or any combination thereof. In some embodiments, the processing device 120 may determine a body shape (e.g., a 3D body shape) of the target subject based on the candidate scan data. The processing device 120 may determine the target water equivalent diameter based on the body shape of the target subject. For example, the processing device 120 may determine a total CT value of the target subject. The total CT value of the target subject may be a sum of CT values of pixels of the target subject in a reconstructed image. The processing device 120 may generate a phantom (e.g., a cylindrical water phantom) with the target water equivalent diameter to represent the target subject based on the total CT value of the target subject. For illustration purposes, the processing device 120 may determine the target water equivalent diameter according to Equation (2):

$${V}_{p} = \frac{π {D}^{2}}{4} * \frac{{V}_{w}}{S * L} , \qquad ( 2 )$$

where Vₚ refers to a total CT value of the target subject; D refers to a target water equivalent diameter of a phantom corresponding to the target subject; V_{w} refers to a CT value of the phantom corresponding to the target subject; S refers to a number (or count) of pixels per inch of a reconstructed image; and L refers to a length of the reconstructed image. The CT value may be used to indicate an average amount of X-ray attenuation associated with a corresponding area of each pixel in a CT image.

In some embodiments, the processing device 120 may determine the target water equivalent diameter based on a topogram image of the target subject. In some embodiments, the topogram image may be associated with a localizer scan. The localizer scan may be performed by a medical device (e.g., the medical device 110) when a radiation source is in a stationary position and a scanning table moves along the Z-axis (e.g., the Z-axis as shown in FIG. 1). For example, if the radiation source is positioned above the object, an anterior-posterior (AP) topogram image may be obtained in a localizer scan. As another example, if the radiation source is positioned on a side of the object, a lateral topogram image may be obtained in a localizer scan. In some embodiments, the topogram image may be used to determine a position of the target subject, a scan angle of the target subject, a layer thickness of a scan region of the target subject, a position relationship between the target subject and the medical device, or the like. The medical device may scan the target subject based on the topogram image. In some embodiments, the body shape of the target subject may be determined based on the topogram image of the target subject. For example, an AP topogram image may be used to estimate the width of the target subject. As another example, a lateral topogram image may be used to estimate the thickness of the target subject. The processing device 120 may determine the target water equivalent diameter of the phantom corresponding to the target subject based on the body shape of the target subject as described elsewhere in the present disclosure.

In 720, the processing device 120 (e.g., the determination module 420) may obtain a plurality of candidate images. Each candidate image may be acquired by a simulated scanning, based on one of a plurality of present values of an imaging scan parameter, of one of a plurality of phantoms of a preset water equivalent diameter.

In some embodiments, the processing device 120 may obtain the plurality of preset water equivalent diameters and a plurality of preset values of the imaging scan parameter. The plurality of preset water equivalent diameters and the plurality of preset values of the imaging scan parameter may be determined manually by a user (e.g., a doctor) of the medical system 100 based on user experience, or be determined by one or more components (e.g., the processing device 120) of the medical system 100 according to different situations. In some embodiments, the plurality of preset water equivalent diameters and the plurality of preset values of the imaging scan parameter may be recorded in a table.

In some embodiments, the processing device 120 may generate the candidate image by performing the simulated scanning on the phantom with the preset water equivalent diameter according to the present value of the imaging scan parameter. For example, the processing device 120 may generate a set of scan data of the phantom with the preset water equivalent diameter based on the present value of the imaging scan parameter according to one or more numerical simulation algorithms. The processing device 120 may generate the candidate image of the phantom based on the set of scan data.

In 730, the processing device 120 (e.g., the determination module 420) may determine a plurality of target values of the imaging scan parameter based on the plurality of candidate images, the plurality of preset water equivalent diameters, and the plurality of preset values of the imaging scan parameter.

In some embodiments, the processing device 120 may evaluate the quality (e.g., the density resolution, the spatial resolution, the signal-to-noise ratio) of the plurality of candidate images of the plurality of phantom. The processing device 120 may select a plurality of target images from the plurality of candidate images. The quality of the plurality of target images may be equal to higher than a quality threshold. The processing device 120 may determine the plurality of target values of the imaging scan parameter of the target subject based on a plurality of preset water equivalent diameters corresponding to the plurality of target images, a plurality of preset values of the imaging scan parameter corresponding to the plurality of target images, and the target water equivalent diameter. Merely by way of example, for a specific target image, the processing device 120 may determine a target value of a tube current based on a preset water equivalent diameter corresponding to the specific target image, a preset value of the tube current corresponding to the specific target image, and the target water equivalent diameter, according to Equation (3):

mAsₚ=mAs_{T}*exp(μ*(D−D_{Ref})*α) (3),

where mAsₚ refers to a target value of the tube current of the target subject; mAs_{T} refers to a preset value of the tube current; μ refers to an absorption coefficient of water; D refers to a target water equivalent diameter; D_{Ref} refers to a preset water equivalent diameter; and a refers to an adjustable parameter. As another example, for a specific target image, the processing device 120 may determine a target value of a tube voltage of the target subject based on a preset water equivalent diameter corresponding to the specific target image, a preset value of the tube voltage corresponding to the specific target image, and the target water equivalent diameter, according to Equation (4):

kVₚ=kV_{T}*exp(μ*(D−D_{Reg})*α) (4),

where kVₚ refers to a target value of the tube voltage of the target subject; kV_{T} refers to a preset value of the tube voltage; μ refers to an absorption coefficient of water; D refers to a target water equivalent diameter; D_{Ref} refers to a preset water equivalent diameter; and a refers to an adjustable parameter.

In 740, the processing device 120 (e.g., the determination module 420) may select a value of the imaging scan parameter from the plurality of target values of the imaging scan parameter.

In some embodiments, the processing device 120 may select a target value of the imaging scan parameter as the value of the imaging scan parameter of the target subject. For example, the processing device 120 may select the smallest target value of the imaging scan parameter as the value of the imaging scan parameter. In some embodiments, the processing device 120 may determine a plurality of radiation doses based on the plurality of target values of the imaging scan parameter. The processing device 120 may select a target value of the imaging scan parameter corresponding to the lowest radiation dose among the plurality of radiation doses as the value of the imaging scan parameter.

It should be noted that the above description regarding the process 700 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more operations may be omitted. For example, operation 740 may be omitted. In operation 730, the processing device 120 may select a target image from the plurality of candidate images. The quality of the target image may be equal to higher than a quality threshold. The processing device 120 may determine a target value of the imaging scan parameter of the target subject based on a preset water equivalent diameter corresponding to the target image, a preset value of the imaging scan parameter corresponding to the target image, and the target water equivalent diameter. The processing device 120 may determine the target value of the imaging scan parameter as the value of the imaging scan parameter of the target subject.

In some embodiments, a patient may register his/her name or ID number before a scan, and the processing device 120 may determine whether a prior information database includes target prior information of the patient based on the name or the ID number of the patient. In response to determining that the prior information database does not include the target prior information of the patient, a value of a procedure parameter of the patient may be determined by a user (e.g., a doctor) of the medical system 100 based on user experience, or be determined by one or more components (e.g., the processing device 120) of the medical system 100 based on a scan protocol of the patient.

In response to determining that the prior information database includes the target prior information of the patient, the processing device 120 may determine whether the target prior information includes a recommended value of the procedure parameter of the patient. In response to determining that the target prior information includes the recommended value of the procedure parameter of the patient, the processing device 120 may determine whether the recommended value of the procedure parameter satisfies a scan condition of the patient. In response to determining that the recommended value of the procedure parameter satisfies the scan condition, the processing device 120 may determine the recommended value of the procedure parameter as the value of the procedure parameter of the patient, as described in connection with FIG. 6. In response to determining that the recommended value of the procedure parameter does not satisfy the scan condition, the processing device 120 may determine a plurality of simulation values of the imaging scan parameter based on a candidate value of the imaging scan parameter (or a recommended value of the imaging scan parameter) in the prior information database, as described in connection with operation 610. The processing device 102 may generate a plurality of simulated images based on the plurality of simulation values of the imaging scan parameter and a plurality of simulation values of the image reconstruction parameter, as described in connection with operation 620. The processing device 102 may determine the value of the procedure parameter based on the plurality of simulated images, as described in connection with operation 630. In response to determining that the target prior information does not include the recommended value of the procedure parameter of the patient, the processing device 120 may determine the value of the procedure parameter based on a plurality of simulated images, as described in connection with FIG. 6.

After the scan is performed on the patient, the value of the procedure parameter of the patient, scan data of the patient, and/or an image of the patient generated based on the scan data may be stored in the prior information database. In some embodiments, a plurality of simulated images may be generated based on a plurality of simulation values of the procedure parameter according to a low-dose simulation algorithm as described in connection with FIG. 6. The plurality of simulated images and the plurality of simulation values of the procedure parameter may also be stored in the prior information database.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this disclosure are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction performing system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2103, Perl, COBOL 2102, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. A method for imaging, implemented on a computing device having one or more processors and one or more storage devices, the method comprising:
obtaining feature information of a target subject;
obtaining target prior information of the target subject based on the feature information of the target subject and a prior information database, wherein the prior information database includes prior information of a plurality of candidate subjects; and
determining, based on the target prior information of the target subject, a value of a procedure parameter that relates to a procedure of the target subject using a medical device.

2. The method of claim 1, wherein the procedure parameter includes an imaging scan parameter and an image reconstruction parameter.

3. The method of claim 2, further comprising:
obtaining scan data of the target subject by causing the medical device to scan the target subject based on a value of the imaging scan parameter; and
generating an image of the target subject based on the scan data and a value of the image reconstruction parameter.

4. The method of claim 2, wherein a first modality corresponding to scan data obtained by of the medical device is the same as a second modality corresponding to the target prior information, and the target prior information includes a candidate value of the imaging scan parameter of the target subject, and the determining, based on the target prior information of the target subject, a value of a procedure parameter comprises:
determining a plurality of simulation values of the imaging scan parameter based on the candidate value of the imaging scan parameter;
generating a plurality of simulated images based on the plurality of simulation values of the imaging scan parameter and a plurality of simulation values of the image reconstruction parameter; and
determining the value of the procedure parameter based on the plurality of simulated images.

5. The method of claim 4, wherein the determining a plurality of simulation values of the imaging scan parameter based on the candidate value of the imaging scan parameter comprises:
obtaining an initial value of the imaging scan parameter;
obtaining an initial image based on the initial value of the imaging scan parameter and the candidate value of the imaging scan parameter using a dose simulation model; and
determining at least one of the plurality of simulation values of the imaging scan parameter based on the initial image and the initial value of the imaging scan parameter.

6. The method of claim 5, wherein the dose simulation model is obtained according to a process including:
obtaining a plurality of training samples each of which includes a sample value of the imaging scan parameter, a sample image corresponding to the sample value of the imaging scan parameter, a reference value of the imaging scan parameter, and a reference image corresponding to the reference value of the imaging scan parameter; and
determining the dose simulation model by training a preliminary model based on the plurality of training samples.

7. The method of claim 4, wherein the target prior information includes candidate scan data corresponding to the candidate value of the imaging scan parameter of the target subject, and the generating a plurality of simulated images based on the plurality of simulation values of the imaging scan parameter and a plurality of simulation values of the image reconstruction parameter comprises:
for each simulation value of the plurality of simulation values of the imaging scan parameter,
determining simulation scan data based on the candidate scan data, the candidate value of the imaging scan parameter, and the simulation value of the imaging scan parameter; and
for each simulation value of the plurality of simulation values of the image reconstruction parameter,
generating a simulated image based on the simulation scan data and the simulation value of the image reconstruction parameter.

8. The method of claim 2, wherein a first modality of the medical device is different from a second modality corresponding to the target prior information, and the obtaining target prior information of the target subject based on the feature information of the target subject and a prior information database comprises:
generating a first image of the first modality based on a second image of the second modality in the target prior information.

9. The method of claim 8, wherein the first modality or the second modality includes at least one of an ultrasound imaging, an X-ray imaging, a computed tomography (CT), a magnetic resonance imaging (MRI), a single photon emission computed tomography (SPECT), or a positron emission tomography (PET).

10. The method of claim 2, wherein the obtaining target prior information of the target subject based on the feature information of the target subject and a prior information database comprises:
determining a dimension of a phantom corresponding to the target subject based on the feature information of the target subject and the prior information database.

11. The method of claim 10, wherein the dimension of the phantom corresponding to the target subject includes a target water equivalent diameter of the phantom corresponding to the target subject.

12. The method of claim 11, wherein the prior information database includes candidate scan data of the target subject, and the determining a dimension of a phantom corresponding to the target subject based on the feature information of the target subject and the prior information database comprises:
determining the target water equivalent diameter based on the candidate scan data of the target subject.

13. The method of claim 11, wherein the prior information database includes a topogram image of the target subject, and the determining a dimension of a phantom corresponding to the target subject based on the feature information of the target subject and the prior information database comprises:
determining the target water equivalent diameter based on the topogram image of the target subject.

14. The method of claim 11, wherein the determining, based on the target prior information of the target subject, a value of a procedure parameter comprises:
obtaining a plurality of candidate images each of which is acquired by a simulated scanning, based on one of a plurality of present values of the imaging scan parameter, of one of a plurality of phantoms of a preset water equivalent diameter;
determining a plurality of target values of the imaging scan parameter based on the plurality of candidate images, a plurality of preset water equivalent diameters, and the plurality of preset values of the imaging scan parameter; and
selecting a value of the imaging scan parameter from the plurality of target values of the imaging scan parameter.

15. The method of claim 1, wherein the target prior information includes a recommended value of the procedure parameter, and the determining, based on the target prior information of the target subject, a value of a procedure parameter comprises:
determining whether the recommended value of the procedure parameter satisfies a scan condition of the target subject; and
in response to determining that the recommended value of the procedure parameter satisfies the scan condition, determining the value of the procedure parameter based on the recommended value of the procedure parameter.

16. The method of claim 1, wherein the prior information database is established based on at least one of feature information of a candidate subject, a historical scan protocol of the candidate subject, a historical value of an imaging scan parameter of the candidate subject, a historical value of an image reconstruction parameter of the candidate subject, historical scan data of the candidate subject, a historical image of the candidate subject, a simulation value of the imaging scan parameter of the candidate subject, a simulation value of the image reconstruction parameter of the candidate subject, simulation scan data of the candidate subject, or a simulated image of the candidate subject.

17. A system for imaging, comprising:
at least one storage device including a set of instructions; and
at least one processor configured to communicate with the at least one storage device, wherein when executing the set of instructions, the at least one processor is configured to direct the system to perform operations including:
obtaining feature information of a target subject;
obtaining target prior information of the target subject based on the feature information of the target subject and a prior information database, wherein the prior information database includes prior information of a plurality of candidate subjects; and
determining, based on the target prior information of the target subject, a value of a procedure parameter that relates to a procedure of the target subject using a medical device, wherein the procedure parameter includes an imaging scan parameter and an image reconstruction parameter;
obtaining scan data of the target subject by causing the medical device to scan the target subject based on a value of the imaging scan parameter; and
generating an image of the target subject based on the scan data and a value of the image reconstruction parameter.

18. The system of claim 17, wherein a first modality corresponding to scan data obtained by the medical device is the same as a second modality corresponding to the target prior information, and the target prior information includes a candidate value of the imaging scan parameter of the target subject, and the determining, based on the target prior information of the target subject, a value of a procedure parameter comprises:
determining a plurality of simulation values of the imaging scan parameter based on the candidate value of the imaging scan parameter;
generating a plurality of simulated images based on the plurality of simulation values of the imaging scan parameter and a plurality of simulation values of the image reconstruction parameter; and
determining the value of the procedure parameter based on the plurality of simulated images.

19. The system of claim 18, wherein the determining a plurality of simulation values of the imaging scan parameter based on the candidate value of the imaging scan parameter comprises:
obtaining an initial value of the imaging scan parameter;
obtaining an initial image based on the initial value of the imaging scan parameter and the candidate value of the imaging scan parameter using a dose simulation model; and
determining at least one of the plurality of simulation values of the imaging scan parameter based on the initial image and the initial value of the imaging scan parameter.

20. A non-transitory computer readable medium, comprising executable instructions that, when executed by at least one processor, direct the at least one processor to perform a method for motion correction, the method comprising:
obtaining feature information of a target subject;
obtaining target prior information of the target subject based on the feature information of the target subject and a prior information database, wherein the prior information database includes prior information of a plurality of candidate subjects; and
determining, based on the target prior information of the target subject, a value of a procedure parameter that relates to a procedure of the target subject using a medical device.
